(19) United States
(12) Patent Application Publication
(10) Pub. No.: US 2021/0269768 A1
(43) Pub. Date: Sep. 2, 2021

(54) NEONATAL STROMAL CELLS HAVING LOW MHC-I EXPRESSION AND USES THEROF

(71) Applicant: VETBIOBANK, Marcy L'Etoile (FR)

(72) Inventors: Rodolphe RAKIC, Lyon (FR); Nathalie SAULNIER, Tassin la Demi Lune (FR); Stéphane MADDENS, Roquefort le Bedoule (FR)

(21) Appl. No.: 17/257,383

(22) PCT Filed: Jul. 2, 2019
(86) PCT No.: PCT/FR2019/051638

§ 371 Date: Dec. 31, 2020

(30) Foreign Application Priority Data
Jul. 2, 2018 (EP) 18305853.6

(51) Int. Cl. C12N 5/073; C12N 5/0775; A61K 35/51; A61K 9/00; A61K 9/08; A61K 35/50; A61P 19/02; A61P 7/00; A61P 1/00

(52) U.S. Cl. CPC C12N 5/0605; C12N 5/0665; A61K 35/51; A61K 9/0019; A61P 1/00; A61K 35/50; A61P 19/02; A61P 7/00; A61K 9/08

(57) ABSTRACT

The invention relates to a population of neonatal stromal cells (NSC) showing low MHC-I (MHC-I^{low}) expression and optionally high CD90 expression, a pharmaceutical composition and an injectable solution comprising said NSC population, as well as a method for obtaining said composition from neonatal tissue. The invention also relates to the advantageous use of said cell population in regenerative, veterinary or human medicine, more specifically in the treatment of osteoarthritis and chronic inflammatory diseases and more generally for treating tissue lesions, degenerative diseases, autoimmune diseases, infectious diseases with or without an inflammatory component, or for treating graft rejection and tumour diseases.

### TECHNICAL FIELD

The invention relates to a population of neonatal stromal cells (NSCs) that can be produced industrially, weakly expressing the type I major histocompatibility complex (referred as low MHC-I or MHC-I^{L}), and optionally strongly CD90 (referred to as high CD90 or CD90^{H}), a composition comprising said population of NSC MHC-I^{L} and optionally CD90^{H} and the method for obtaining this composition from neonatal tissues. In particular, the invention relates to the advantageous use of this population of cells in regenerative, veterinary or human medicine or more precisely as treatment of osteoarthritis and chronic inflammatory diseases and more generally for the treatment of tissue lesions, degenerative diseases, autoimmune diseases, infectious diseases with or without an inflammatory component or in graft rejection and tumour diseases. The invention also relates to a ready-to-use pharmaceutical composition comprising such an NSC population.

### CONTEXT

Mesenchymal stromal cells (MSCs) have been isolated for the first time in bone marrow; however, the frequency thereof is fairly low in this tissue and declines with age (0.01% of total mononucleated cells in adults) (Bruder et al., 1997). Cells having similar characteristics have subsequently been identified in the stromal vascular fraction of adipose tissue in higher proportions. MSCs are characterised by the expression of a panel of surface markers CD105, CD73, CD90 and do not express CD45, CD34, CD14 or CD11b, CD79alpha or CD19 and HLA-DR (Dominici, 2006). Moreover, MSCs must be capable of differentiating in adipogenic, osteogenic and chondrogenic lineages in vitro, using adapted differentiation media. In the context of therapeutic use, MSCs are normally isolated from an adult tissue (bone marrow, adipose tissue) taken from a patient, and reinjected into the patient himself following an amplification phase in the laboratory. A delay is therefore necessary administering the therapy to the patient.

MSC populations isolated in a conventional way from a plurality of samples coming from the same tissue source but different subjects are mutually heterogeneous (inter-population heterogeneity) from the point of view of the cell markers. In addition, it is also considered that, within a cell population coming from the same sample, several MSC phenotypes coexist (intra-population heterogeneity). This implies a lack of reproducibility in the context of industrial use thereof and clinical efficacy (Phinney, 2012). This is because the cell phenotype influences the biological properties and characteristics. This inter- and intra-population heterogeneity is in particular found for MHC-I and CD90 membrane markers present in MSCs. Several studies show variations in the expression of these markers according to the tissue sources and isolation or amplification methods.

Jacobs et al (2013) mention multi-potent human adult progenitor cells isolated from bone marrow, having low expression of MHC-I and high expression of CD90, capable in particular of differentiating into osteoblasts and chondrocytes.

Tessier et al (2015) and Davies et al (WO 2004/072273) described neonatal MSCs weakly expressing MHC-I and expressing CD90, but nevertheless the work by these teams shows both a high fluctuation of the expression of MHC-I among strains isolated during the cryopreservation and amplification process (Lepage et al., 2019) (Sarugaser, et al., 2005). Portmann-Lanz et al., (2006) described MSCs isolated from foetal membranes and human placenta tissues and show a high variability in the expression of CD90 and MHC-I in these MSCs.

These various works descriptively use the expression of MHC-I without identifying the effects of these differences in expression on the biological properties of MSCs. The taking into account of the fluctuation of these two markers, MHC-I and CD90, and the functional characteristics associated with MSCs were not investigated.

Some tissue sources such as the placenta show high differentials in expression of these markers, thus limiting the reproducibility of a strategy for industrial use and clinical application.

Moreover, it should be noted that a high technical constraint lies in the methods for analysing the expression of MHC-I and/or CD90 in these studies. This is because all these works define their cells as being positive or negative for these markers. This concept of positivity or negativity requires the determination of a threshold, the relevance of which is dependent on many technical and biological parameters. This involves among other things analysis limits for the heterogeneous MSC populations and/or for the weakly expressed markers.

None of the characteristics of the cells described in these documents makes it possible to envisage reproducible large-scale culture and therefore industrial application of the cell production, in particular for therapeutic use of these cells.

### OBJECTIVES

One objective is to provide a population of cells intended for cell therapy, having good capacity for proliferation in order to enable it to be produced on an industrial scale, and this in a reproducible fashion. Another objective is to provide a population of cells intended for cell therapy, having abilities to differentiate in other types of cell, in particular chondrocytes, while limiting the risk of formation of ectopic tissue. Another objective is to provide cells capable of interacting with the cells of the immune system and regulating their activity. Another objective is to provide novel means for effective treatments of tissue lesions, degenerative diseases, auto-immune diseases, diseases with an inflammatory component and infectious diseases, or in graft rejection and tumour diseases, in particular in non-human mammals, especially dogs, horses or cats, or in humans.

Yet another objective consists of providing a method for obtaining the cell population meeting the criteria sought, from samples taken non-invasively, in order to allow industrial production, and this in a reproducible manner.

The inventors discovered surprisingly that neonatal tissues comprised a sub-population of NSCs of MHC-I^{L} phenotype and optionally CD90^{H} having a high potential for proliferation and thus enabling industrial use thereof and advantageous use thereof in cell therapy, in particular for treating tissue lesions and degenerative diseases in veterinary or human medicine.

The use of these cells is all the more justified for the treatment of joint pathologies, in the light of their high potential for chondrogenic differentiation, their low potential for osteogenic differentiation and their immunomodulation capability. Moreover, the under-expression of MHC-I among these cells could induce a limited immune response vis-à-vis these cells, compared with that induced vis-à-vis cells strongly expressing MHC-I.

The inventors have also succeeded in overcoming the high inter- and intra-population heterogeneity existing in the placenta in order to provide a homogeneous population of placenta CMH-1^{L} and optionally CD90^{H} NSCs having a high proliferation potential and thus enabling them to be used industrially and to be used advantageously in cell therapy.

In addition, the biological properties of the NSCs isolated by the inventors remains stable during cryopreservation/thawing steps.

### DETAILED DESCRIPTION

Population of NSCs

A first object of the present disclosure relates to a population of neonatal stromal cells (NSCs), comprising neonatal stromal cells of MHC-I^{L} phenotype, and optionally of CD90^{H} phenotype. The inventors discovered that these cells isolated from a neonatal biological sample had a high capacity for multiplication, thus conferring thereon a high potential for industrial use and strong therapeutic potential.

MHC-I corresponds to the class 1 major histocompatibility complex, expressed almost ubiquitously in the organism.

MHC-I, also referred to as HLA-I (human leukocyte antigen class I) in humans, results from the expression of a family of genes referred to as HLA genes. Among these genes, mention can be made of HLA-A, HLA-B and HLA-C, which code for the conventional forms of HLA-I. In dogs, MHC is also referred to as DLA (Dog Leukocyte Antigen), in horses ELA (Equine Leukocyte Antigen) is spoken of, and in cats FLA (Feline Leukocyte Antigen) is spoken of.

CD90 corresponds to the membrane protein “Thy-1 Cell Surface Antigen” and is expressed by a great majority of the cells of the stroma.

“MHC-I^{L} phenotype” means that the NSCs have a very weak expression, or an absence of expression, of MHC-I. “CD90^{H} phenotype” means that the NSCs have a strong expression of the surface antigen CD90.

The cells according to the present disclosure are naturally of low MHC-I phenotype (MHC-I^{L}) and optionally high CD90 (CD90^{H}), that is to say the low expression of MHC-I and the high expression of CD90 is not a phenotype resulting from genetic modification on the population of cells.

In a particular embodiment, the population of NSCs comprises neonatal cells of MHC-I^{L} phenotype and of CD90^{H} phenotype (denoted MHC-I^{L}/CD90^{H}).

The expression of these two markers in the cell population can be evaluated by the flow cytometry technique.

A cell population may be termed homogeneous when a population of NSCs (isolated from a single donor source) is homogeneous from a point of view of the MHC-I and CD90 expression (absence of intra-population heterogeneity), that is to say the cells all have a similar degree of expression of MHC-I and of CD90, and therefore all have the same phenotype vis-à-vis the expression of the MHC-I and CD90 markers (FIG. 12).

A cell population is termed heterogeneous when a population of NSCs (isolated from a single donor source) is composed of various sub-populations expressing MHC-I and/or CD90 differently. Intra-population heterogeneity is spoken of.

In the case of a homogeneous population, analysis of the expression of the MHC-I and CD90 markers can be done in flow cytometry by a so-called single marking strategy, that is to say the marking of each of the markers is done independently in two separate tubes.

In the case of a heterogeneous population, analysis of the expression of the MHC-I and CD90 markers can be done in flow cytometry by a double marking, that is to say a simultaneous marking of the two markers in a single tube for the same cell population in order to clearly distinguish the sub-populations. Double marking may also be used in the case of a homogeneous population.

When a population of NSCs comprising cells with an MHC-I^{L}/CD90^{H} phenotype of interest is considered, said population is considered to be heterogeneous when said population of NSCs comprises between 10% and 90% cells of MHC-I^{L}/CD90^{H} phenotype.

When a population of NSCs comprising cells of MHC-I^{L}/CD90^{H} phenotype of interest, said population is considered to be homogeneous when said population of NSCs comprises more than 90% cells of MHC-I^{L}/CD90^{H} phenotype or less than 10% cells of MHC-I^{H}/CD90^{L} phenotype.

The level of expression of a marker (low or high or absence of expression) can be assessed by an analysis technique by flow cytometry correctly set up by a person skilled in the art. The concept of low or high expression implies a concept of discrimination threshold making it possible to correctly discern this population from populations having conversely a high or low expression of said marker from an analytical point of view. The methodology for analysis of expression of a marker is dependent on the marking strategy for the marker used. For example, the use of fluorochrome with different fluorescence yields will not have the same detection ability. The use of marking strategies with signal amplification systems (biotin-avidin for example) will also have an influence on the capabilities for detection of the antibody of interest. It is therefore essential to define suitable positivity/negativity thresholds according to the strategy used.

The expression of MHC-I in particular, in NSCs, is relatively low compared with other types of cell such as fibroblasts or peripheral blood mononucleated cells (PBMCs). The inventors have therefore developed a strategy for marking MHC-I that is fairly sensitive for detecting small differences in the expression of this marker in NSCs.

The results in FIGS. 12 and 14 show that analysis of MHC-I and CD90 by single marking can show itself to be limiting in the case of intra-population heterogeneity. Analysis of fluorescence averages for heterogeneous populations does not make it possible to establish with precision whether a total population is of MHC-I^{L}/CD90^{H} or MHCI^{H}/CD90^{L} phenotype. The double-marking methodology thus affords better resolution in the context of the characterisation of NSC population showing high intra-population heterogeneity.

Double Marking of the MHC-I and CD90 Markers

An example of simultaneous marking (also referred to as immunophenotypical double marking or co-marking) of MHC-I and CD90 in the same NSC population can be implemented using:
for marking of MHC-I, a primary anti-mouse MHC-I IgG2a antibody, such as the primary anti-MHC-I DG-BOV2001/DG-H58A IgG2a antibody (Monoclonal Antibody Center Washington State University), revealed with a secondary goat F(ab′)2 secondary anti-mouse IgG antibody coupled to allophycocyanin (APC), and
for marking CD90, an anti-CD90 monoclonal antibody coupled to phycoerythrin (PE), such as the monoclonal anti-rat CD90/Thy1 antibody Antibody YKIX337.217 (PE).

After marking, the fluorescence of the APC and PE fluorochromes is analysed. In order to discern the subpopulations of NSC, a 2D representation of the APC and PE fluorescence is produced. The results are compared with those obtained with the use of the isotypes coupled to the respective fluorochromes described previously.

A population or sub-population of NSC is termed MHC-I^{L}/CD90^{H} if the ratio of the averages of the fluorescence intensity mean fluorescence intensity (MFI) between the MHC-I and its control isotype (also referred to as relative MFI or rMFI) is below a threshold of 20, more particularly 15, more particularly 10, and if the rMFI between the CD90 marker and its control isotype is above 15, more particularly 20.

A population or sub-population of NSC is termed MHC-I^{H}/CD90^{L} if the ratio of the mean fluorescence intensity (MFI) between the MHC-I and its control isotype is above 20, more particularly 15, more particularly 10, and if the rMFI between the CD90 marker and its control isotype is below 15, more particularly 20.

With double marking, the presence of various NSC sub-population profiles with regard to the expression of the MHC-I/CD90 markers (CMH-I^{L}, CMH-I^{H} and CD90^{H}, CD90^{L}) is revealed, in the same cell population coming from the same sample (FIG. 12). These various phenotypes in the expression of the MHC-I/CD90 markers involve biological differences between the various sub-populations revealed.

Analysis of the expression of the two MHC-I and CD90 markers can be carried out for a plurality of populations of NSC issuing from sources defined and isolated according to the same experimental protocol. The inventors have confirmed by their method the existence of an inter-population heterogeneity vis-à-vis the expression of these two markers among the NSC populations coming from neonatal tissues of various natures (placenta, umbilical cord, umbilical cord blood) (FIG. 13).

Single Marking of the MHC-I and CD90 Markers

Alternatively, a single marking of MHC-I and CD90 can be carried out.

An example of single marking of MHC-I in an NSC population, for cytometric analysis, can be produced using the primary anti-MHC-I DG-BOV2001/DG-H58A IgG2a antibody (Monoclonal Antibody Center Washington State University) and the control isotype primary antibody Mouse-COL2002/COLIS205C IgG2a (Monoclonal Antibody Center Washington State University). These antibodies that are not coupled to a fluorochrome are used conjointly with the use of a secondary rabbit F(ab′)2 anti-mouse IgG STAR12A antibody (AbSerotec) marked by phycoerythrin (PE). This cytometric analysis method is described in more detail in Example B. 2).

In this embodiment of single marking, a population of NSCs is considered to be the low MHC-I (MHC-I^{L}) phenotype, if the ratio between the mean fluorescence intensity (MFI) values as measured in flow cytometry of the cells marked by a specific antibody of an epitope of MHC-I with respect to the MFI values of the cells marked by the control isotype is less than 3, in particular less than 2.5, in particular less than 2, more particularly less than 1.96.

Conversely, a population of NSCs is considered to be of high MHC-I (MHC-I^{H}) phenotype, according to the same method, if the ratio between the MFI values, as measured in flow cytometry, of the cells marked by a specific antibody of an epitope of MHC-I with respect to the MFI values of the cells marked by the control isotype is greater than 1.96, in particular greater than 2, in particular greater than 2.5, more particularly greater than 3.

In another embodiment, the single marking of MHC-I within a population of NSCs, for a cytometric analysis, can be carried out using the primary anti-MHC-I DG-BOV2001/DG-H58A IgG2a antibody (Monoclonal Antibody Center Washington State University) and the control isotype primary antibody Mouse-COL2002/COLIS205C IgG2a (Monoclonal Antibody Center Washington State University). These antibodies that are not coupled to a fluorochrome are used conjointly with the use a secondary goat F(ab′)2 secondary anti-mouse IgG antibody (eBioscience) coupled to allophycocyanin (APC). This cytometric analysis method is described in more detail in the Example B. 2 part).

In this embodiment of single marking, an NSC population is considered to be the MHC-I^{L} phenotype if the ratio between the mean fluorescence intensity (MFI) values as measured in flow cytometry of the cells marked by a specific antibody of an epitope of MHC-I with respect to the MFI values of the cells marked by the control isotype is below 12, in particular below 10, more particularly below 9.

Conversely, an NSC population is considered to be MHC-1^{H}, according to this method, if the ratio between the MFI values as measured in flow cytometry of the cells marked by a specific antibody of an epitope of MHC-I with respect to the MFI values of the cells marked by the control isotype is greater than 9, in particular greater than 10, more particularly greater than 12.

In another embodiment of single marking, an NSC population is considered to be the MHC-I^{L} phenotype if the ratio between the mean fluorescence intensity (MFI) values as measured in flow cytometry of the cells marked by a specific antibody of an epitope of MHC-I with respect to the MFI values of cells marked by the control isotype is less than 20, in particular less than 15, more particularly less than 10.

The determination of these thresholds is explained in Example A, part 2) e) and in FIG. 3.

Conversely, an NSC population is considered to be the MHC-I^{H} phenotype if the ratio between the mean fluorescence intensity (MFI) values as measured in flow cytometry of the cells marked by a specific antibody of an epitope of MHC-I with respect to the MFI values of cells marked by the control isotype is greater than 20, in particular greater than 15, more particularly greater than 10.

Furthermore, the method based on flow cytometry, analysis of the MHC-I phenotype can also be confirmed at the protein and/or transcription level.

At the protein level the expression of MHC-I, whatever the species of interest, can be studied by the use of antibodies or fragments of antibodies (monoclonal or polyclonal) specifically directed against one or more epitopes of HLA-A, B, C, or homologues thereof according to the species. The techniques associated with these antibodies comprise among other things cytometric analysis, Western blot, the ELISA test, immunofluorescence and/or immunohistochemistry. More generally, technologies involving interaction between MHC-I and a marked protein, such as an inhibitor, can be used as analysis tools. Other technologies using oligonucleotides marked by a probe can serve to evaluate this expression, such as the use of aptamers, RNA probes and/or DNA probes.

At the transcription level, analysis of the expression of the various genes can be done by end-point PCR, RTqPCR, digital PCR and/or microarray RNA. It is also possible to measure the transcriptional expression of a component of the molecules of MHC, as beta 2 microglobulin (B2M) may be by way of example.

With regard to single marking of CD90 for flow cytometry analysis, this can for example be done using an anti-CD90 antibody coupled to the fluorochrome PE, such as the CD90 antibody Antibody YKIX337.217 (Bio Rad) with respect to the signal obtained by an isotypical control coupled to the same fluorochrome Mouse (BALB/c) IgG1,κ MOPC-21 (AbSerotec). In this embodiment, a population of NSCs is considered to be the high CD90 phenotype or so-called CD90 (CD90^{H}), if the ratio between the mean fluorescence intensity (MFI) values as measured in flow cytometry of the cells marked by a specific antibody of an epitope of CD90 with respect to the MFI values of the cells marked by the control isotype is greater than 15, more particularly greater than 20 (Example B part 2)).

Conversely, a population of NSCs is considered to be the phenotype CD90^{L}, if the ratio between the mean fluorescence intensity (MFI) values as measured in flow cytometry of the cells marked by a specific antibody of an epitope of MHC-I with respect to the MFI values of cells marked by the control isotope is less than 15, more particularly less than 20. Furthermore, the method based on flow cytometry, analysis of the CD90 phenotype can also be confirmed at the protein and/or transcription level using antibodies specifically directed against one or more epitopes of CD90 and/or at the transcription level by analysis of the Thy1 gene coding for CD90 by end-point PCR techniques, RT-qPCR, digital PCR and/or RNA microarray.

Determination of the Threshold

Whether by single or double marking, evaluation of the level of expression of MHC-I and CD90 requires the determination of a threshold. “Determination of the threshold” means the methodology that a person skilled in the art can apply in order to reveal all the differential expressions of MHC-I and/or of CD90 among the populations of cells and more particularly of NSCs and to define therefrom an acceptable relative expression limit. The role of this threshold is to exclude NSCs overexpressing MCH-I and/or underexpressing CD90 and to exclude those underexpressing MHC-I and/or overexpressing CD90 in the industrial production procedure.

The majority of works on MSCs described in the prior art use the expression of membrane markers by means of the concept of positivity or negativity of a cell for a given marker (that is to say the absence or presence of this marker). These methodological thresholds are determined from the population of NSCs the non-specific epitopes of which have been saturated and marked by isotypical antibodies not targeting any epitopes specifically (isotypical population). The positivity/negativity thresholds are fixed generally at between 2 and 3 standard deviations of the isotypical population the statistical distribution of which is of a Gaussian nature. That is to say a threshold placed so that 95.45%-99.73% of the isotypical population corresponds to the negative population of the cells marked for the marker of interest. For the highly specific protein markers of a particular cell type, this threshold plays a role in the interpretation of the data.

However, in the case of MHC-I and CD90, these two proteins are expressed by a great majority of the cells of the stroma and are not specific to the MSCs or NSCs. There exists moreover a fluctuation in expression that is greater or lesser between the various cell types, including among the MSCs and the NSCs. Thus, placing the positivity/negativity thresholds between 2 or 3 standard deviations of the isotypical population may give rise to a significant methodological bias for low expressions, such as for the analysis of NSCs described in the present invention.

Thus, despite the technical possibility of evaluating the positivity or negativity of cells for MHC-I and/or CD90, it is possible that the criterion for belonging to these categories does not represent biological reality. This is because the MHC-I^{L} and/or CD90^{L} character reflects merely a low expression of this marker by the cells and implies a relative positivity for all the cells (FIG. 1E and FIG. 14).

Ideally and in the context of a marking strategy for a given panel of antibodies and for analysis of MHC-I and/or CD90, a precise threshold can be determined by a person skilled in the art by means of the analysis of a statistically sufficient number, or representative sample, of cell population belonging to the two categories: underexpressing MHC-I and overexpressing MHC-I by way of example which, respectively, allow industrial use of NSCs or do not allow it.

The character of industrial use, which is coupled to the low expression of MHC-I and optionally of the MHC-I^{L}/CD90^{H} phenotype in order to discern the two populations of NSC, may be replaced by another biological character insofar as the latter can be correlated with the expression of MHC-I and/or of CD90 (e.g. the character of chondrogenic differentiation).

The obtaining, by a person skilled in the art, of this sufficiently large number of populations of NSCs, or representative sample, from a statistical point of view in the two categories of NSC makes it possible to determine a discrimination limit between the two populations. In the context of the invention, this discrimination limit results in the determination of a threshold of relative expression of MHC-I and/or of CD90 and more precisely in the context of cytometric analysis by a threshold of rMFI.

“Discrimination limit” means the threshold and the fluctuations of this threshold making it possible unambiguously to distinguish the NSCs overexpressing MHC-I or CD90 and underexpressing MHC-I or CD90. This discrimination limit must make it possible to validate high expression for MHC-I or low expression for CD90 of a population of NSCs that cannot be used industrially with high sensitivity, that is to say to guarantee a high interpretation as a true positive. Moreover, this discrimination limit must make it possible to validate low expression for MHC-I or high expression for CD90 of a population of NSCs that can be used industrially with high specificity, that is to say to guarantee low interpretation as a false negative. Insofar as the number of populations of NSCs analysed is sufficiently great, a person skilled in the art can refine this discrimination limit by relying on the curve of the efficacy function of the receptor (ROC curve).

Analysis of the MHC-I has several repercussions from an industrial point of view since the expression thereof is firstly specific to the tissue origin but also dependent on the microenvironment of the NSCs in vivo and/or in vitro.

From a technical point of view, this discrimination limit, or threshold, can serve firstly to exclude industrial use of the cells that cannot be used industrially and secondly:
The NSCs that have already undergone stimulation to an in vivo or ex vitro inflammatory environment;
The NSCs contaminated by an NSC population not meeting the selection criteria;
The NSCs contaminated by a population of another cell type expressing high levels of MHC-I (for example, fibroblasts).

Concerning CD90, the expression of this is dependent on the state of differentiation of the cells and serves as an indicator for a biological aspect other than those taught by the expression of MHC-I (Sibov et al., 2012). It is possible for a person skilled in the art to establish an analysis method similar to that described for MHC-I.

It is in fact advantageous to use an immunological marking strategy making it possible to assess the expression panel of these two markers.

The concept of “panel” means, by order of magnitude, at a minimum 2 categories of expressions observable in various cell types and phenotypes of NSC analysed in our experiments for MHC-I and for CD90 (Table 1).

TABLE 1
Categories of observable expressions of the markers MHC-I and
CD90 in NSCs that can be used industrially, NSCs that cannot be
used industrially, NSCs stimulated by IFN-γ and fibroblasts.

| | NSCs that can | NSCs that | NSCs | |
|---|---|---|---|---|
| | be used | cannot be used | stimulated by | |
| Marker | industrially | industrially | IFN-γ | Fibroblasts |
| MHC-I | Low | High | High | High |
| CD90 | High | Low | Variable | Variable |

The discrimination limit may vary according to the analysis technique used or the parameterising of the analysis tools used. By way of example, in the case of a fluorescence cytometry analysis, it is possible to increase the acquisition signal during the cytometry analysis and to vary the positivity/negativity thresholds by:
an increase in the sensitivity of the photomultipliers,
use of secondary antibodies,
use of polyclonal primary and/or secondary antibodies and/or cocktails of monoclonal antibodies and/or having a better titre for the epitope of interest,
an antibody with a high protein/fluorochrome ratio,
an antibody coupled to a fluorochrome with high emission energy.

Thus, in order to enable populations of interest to be selected, a person skilled in the art can finely determine the discrimination limit according to the following three aspects: the given analysis strategy, the given marking strategy and/or the parameterising of the given analysis tool. In the context of the analysis strategy used by the inventors, the methodology is detailed in the material and method and in the figures associated with this document.

The present invention therefore relates to a population of NSCs comprising NSCs of MHC-I^{L} phenotype, and optionally of CD90^{H} phenotype.

“Population de cells” means a set of cells comprising one or more different cell types, for example a mixture of cells at different differentiation stages, comprising cells having the same tissue origin or issuing from various tissue origins.

According to one embodiment, the population of NSCs comes from a neonatal tissue sample, in particular from one or more placentas and/or from one or more umbilical cords, and/or from one or more amniotic membranes, or from a sample of neonatal fluid, in particular the blood of one or more umbilical cords, or the amniotic liquid of one or more amniotic fluids.

The advantage of neonatal tissues makes it possible to have available a large number of MHC-I^{L}/CD90^{H} NSCs from one sample.

For this purpose, the extra-embryo [embryonic??] appendages (placenta, umbilical cord, amniotic membrane) are generally taken aseptically during caesareans or by natural parturition in gestating females, preferably at term. For example, as soon as the newborn baby has left the amniotic sac and is made safe, the extra-embryonic tissue is immediately transferred into a transport box containing for example Dulbecco's phosphate-buffered saline solution in order to be taken to the laboratory. As for umbilical cord blood, this can be recovered by puncture at the umbilical vein, in particular using a needle connected to a blood-sampling pouch or a tube or any other container.

In another example, the amniotic liquid can be recovered by puncture through the amniotic membrane, in particular using a needle connected to a blood-sampling pouch or a tube or any other container.

Ratio of the NSCs of Interest in the Cell Population and Origin of NSCs

In a particular embodiment, the population of NSCs according to the invention is characterised in that less than 20% in number, in particular less than 15%, more particularly less than 10% of the cells of said population are MHC-I^{H} and optionally characterised in that less than 20% in number, in particular less than 15%, more particularly less than 10% of the cells of said population are CD90^{L}.

In a particular embodiment, the population of NSCs according to the invention is characterised in that it comprises at least 80% in number, in particular at least 85%, more particularly at least 90% of cells of MHC-I^{L}/CD90^{H} phenotype.

The population of NSCs according to the invention therefore corresponds either to a homogeneous population of NSCs of MHC-I^{L}/CD90^{H} phenotype, that is to say a population comprising between 90 and 100% NSCs of MHC-I^{L}/CD90^{H} phenotype, or a heterogeneous population comprising between 80 and 90% NSCs of MHC-I^{L}/CD90^{H} phenotype (FIG. 13).

In a particular embodiment, the NSCs come from a neonatal tissue sample, such as one or more placentas and/or one or more umbilical cords, or one or more amniotic membranes, or a sample of neonatal fluid such as for example the amniotic liquid from one or more amniotic sacs and in particular the blood of one or more umbilical cords.

In a particular embodiment, the population of neonatal stromal cells, comprising neonatal stromal cells of MHC-I^{L} phenotype, and optionally of CD90^{H} phenotype, is a population of NSCs issuing from one or more placentas.

In a particular embodiment, said population of NSCs is a population of placental NSCs and comprises at least 80% in number, in particular at least 85%, more particularly at least 90% cells of MHC-I^{L}/CD90^{H} phenotype.

In a particular embodiment, the sample comes from neonatal tissues or fluids of a mammal, and in particular of a dog, a cat, a horse or a human.

In a more particular embodiment, the neonatal sample comes from a dog or a cat.

Other Structural Characteristics of the NSCs of Interest

The NSCs may furthermore be characterised by the fact that fewer than 10% of the cells express one or more of the following surface markers: CD11b, CD14, CD31, CD34, CD45, or HLA-DR, also called MHC-II.

The NSCs may also be characterised by the expression, at transcription level, of growth factors, such as the vascular endothelium growth factor (VEGF), the growth factor (HGF), the keratinocyte growth factor (KGF), and the type B transforming growth factor (TGF-β).

Biological Characteristics of the NSCs According to the Invention

Apart from their structural properties, the NSCs according to the present disclosure can be characterised by their biological characteristics.

This is because the NSCs according to the invention can be characterised by their cell proliferation capacity.

Thus, in a specific embodiment, the population of NSCs is characterised in that at least 80% of the cells of said population of NSCs have a consecutive cell doubling capacity greater than 20 total doublings.

“Cell proliferation capacity” means that a population of NSCs according to the invention is capable of doubling in number, more than 20 times in total.

In order to evaluate the number of doublings, at each cell passaging, the cells are detached from their support, centrifuged and then counted, for example by the trypan blue exclusion technique using an electronic counter. The number of doublings at each cell passaging is calculated in accordance with the following formula: Number of doublings=LOG (Nf/Ni)/LOG(2) (Nf: number of final cells and Ni: number of initial cells). The total number of cell doublings is equal to the sum of the number of total doublings at each cell passaging.

The cells according to the present disclosure can be used industrially, that is to say the original source thereof can supply a large quantity of cells of interest and make possible in vitro cell amplification on an industrial scale. According to one embodiment, the NSCs have a proliferation capacity greater than twenty total cell doublings during at a minimum four cell passagings.

Among various populations of NSCs isolated from the same source and more particularly from canine placenta, the inventors have shown that there exists a disparity in proliferation potential, making it difficult to use some of these NSC populations industrially (FIG. 15). The inventors have completely unexpectedly identified that the MHC-I^{L}/CD90^{H} phenotype of the NSCs was related to the proliferation capacity of the cells, enabling them to double a larger number of times in vitro than the NSCs of MHC-I^{H}/CD90^{L} phenotype (FIG. 4). This capacity for cell doublings makes it possible thereby to achieve a sufficient number of cells for producing therapeutic preparations and thus to mass produce these cells for use in particular in veterinary or human medicine. Moreover, this doubling capacity has a limit, thus minimising genetic drift of the cells and the emergence of a neoplasic phenotype. This characteristic is an additional argument for the use of these NSCs as a cell or tissue therapy product.

The population of neonatal stromal cells can also be characterised in that at least 80% of the cells of said population of NSCs have:
an ability to adhere to the plastic support; and/or
a potential for chondrogenic differentiation, and/or
an immunomodulating potential.

In a specific embodiment, the population of neonatal stromal cells is also characterised in that at least 80% of the cells of said population of NSCs have a limited osteogenic differentiation potential or do not have any osteogenic differentiation potential.

“Ability to adhere to plastic support” means that the population of NSCs is characterised by its property of adhesion to a plastic support.

“Potential for chondrogenic differentiation” means that the NSCs have the ability to be able to differentiate into chondrocytes. The expressions “chondrocyte differentiation” and “differentiation into chondrocytes” can also be used indifferently.

This capacity for differentiation into chondrocytes can be assessed by a protein and/or transcription study of the specific markers of cartilage such as collagen of type II (COL2A1), SOX-9 (SOX9), aggrecan (ACAN), the cartilage Oligomeric Matrix Protein (COMP), the type IX collagen (COL9A1), the type XI collagen (COL11A1), the type collagen IIB (COL2B) after induction of chondrogenesis. Moreover, viscoelastic properties of the extracellular matrix, expressed by the differentiated NSCs, are similar to the viscoelastic properties of cartilage. These viscoelastic properties can thus serve as a characteristic for evaluating chondrogenesis.

In a privileged culture mode, in order to induce chondrogenesis, the NSCs are detached from their support by trypsination and used to form micromasses by gravitation in a drop of amplification medium such as for example DMEM (1 g/l of glucose) supplemented with 10% SVF (vol:vol), 2 mM of glutamine and 0 to 20 ng/ml of fibroblastic growth factor (FGF2-β). A micromass of NSC then forms after 24 hours of incubation, at the base of the drop. This micromass is recovered and put in the presence of a chondrocyte differentiation medium composed of DMEM with 4.5 g/l of glucose and with TGF-β3 or a TGF-β1/BMP-2 combination added, during 7 to 28 days. At the end, an RNA or protein extraction is performed in order to analyse the expression of specific markers of the chondrogenic lineage such as type II collagen, aggrecan, COMP or SOX9.

The MHC-I^{L}/CD90^{H} NSCs have an increased chondrocyte differentiation potential compared with MHC-I^{H}/CD90^{L} NSCs with regard to the size of the micromasses generated (see FIGS. 5A, 5B, 5C and 5D) and/or the expression of chondrogenic markers such as COL2A1, SOX9, as well as with regard to the expression of a sulfated extracellular matrix composed of type II collagen. This relationship is in accordance with the advantageous therapeutic use of the NSCs according to the present disclosure in the arthropathy context since these can participate in the tissue repair of articular cartilage.

The inventors have shown that the MHC-I^{L}/CD90^{H} phenotype of the NSCs according to the present disclosure can be correlated with a high capacity for chondrogenic differentiation of the cells in comparison with cells of the MSC type, issuing from adipose tissue, from amnion or from bone marrow, also having this chondrogenic ability.

Thus, in a particular embodiment, the population of MHC-I^{L}/CD90^{H} NSCs according to the invention is characterised in that at least 80% of the cells of said population of NSCs have a chondrogenic differentiation potential. In particular, this chondrogenic differentiation potential is assessed with respect to the expression of the Col2a1 gene, and is at least 10 times greater compared with a population of MHC-I^{H}/CD90^{L} NSCs.

“Immunomodulator potential” means the capacity for immunomodulation of NSCs.

This potential can be characterised by the capacity that NSCs have for expressing a set of immunomodulator factors such as PGE2, IL-6, IL-10, TGF-β, IDO, iNOS, HGF, KGF, CCL2 and/or TSG-6. This is because, in the presence of an inflammatory context, NSCs can modify their phenotype. This inflammatory context can be mimed in vitro by stimulation of cells by means of cytokines and/or growth factors such as IFN-γ, IL-1, IL-6 and/or TNF-α. A modification of the phenotype of the NSCs results in the ability of the NSCs to modify the transcription and/or protein expression of markers involved in immunomodulation. Among these markers mention can be made of PGE2, IL-6, IL-10, TGF-β, IDO, iNOS, HGF, KGF, TSG-6, CCL2, CMH-I, CMH-II, HLA-E. Thus, the immunomodulating potential can for example be determined by a study of the expression of the prostaglandin (PGE2) secreted by the cells in basal condition and after stimulation in an inflammatory context. For this the cells are cultivated in a proliferation medium (medium with foetal calf serum) or in a medium complemented with gamma interferon, and then the PGE2 secreted is measured by ELISA test (FIG. 6).

The immunomodulation potential of NSCs can also be characterised by the antiproliferation effect of the NSCs on peripheral blood mononucleated cells (PBMCs) treated with a mitogenic agent such as phytohaemagglutinin, concanavalin A and/or lipopolysaccharides. The immunomodulation of the NSCs can also be evaluated by the ability of the NSCs to inhibit the proliferation and secretion of pro-inflammatory cytokines and/or the differentiation of the T and NK lymphocytes, B lymphocytes, monocytes and/or macrophages.

Thus the immunomodulating potential of NSCs can also be determined by the ability of the NSCs to inhibit the proliferation of lymphocytes in vitro. For this purpose blood mononucleated cells (PBMCs) previously incubated with a fluorescent dye are co-cultivated with the NSCs in the NSC/PBMC proportion 1:10 in the presence of a mitogenic agent such as concanavalin A. After 4 days of culture at 37° C., the non-adherent cells are recovered and marked by an anti-CD3 antibody (specific to T lymphocytes) coupled to a fluorochrome, and then analysed in flow cytometry in order to evaluate the signal of the fluorescent dye within the positive CD3 population. The comparison of the marking with that achieved with a control corresponding to the PBMCs cultivated in the presence of mitogenic agent but without NSC (PBMC-ctrl) makes it possible to define a proliferation index for the lymphocyte population. With a (NSC+PBMC 1:10)/(PBMC-ctrl) ratio of less than or equal to 0.5, the NSCs are considered to be exerting a significant antiproliferative activity (FIG. 7 and FIG. 8D).

The inventors have in fact shown that the NSCs of MCH-I^{L}/CD90^{H} phenotype according to the present disclosure have good immunomodulation capability.

In a specific embodiment, the population of NSCs according to the present disclosure can also be characterised in that at least 80% of the cells of said population of NSCs do not have any osteogenic differentiation potential. Low osteogenic differentiation potential or low osteogenesis potential are also spoken of.

This absence of osteogenic differentiation potential or low osteogenic differentiation potential can be evaluated by the protein and/or transcription study of specific markers of the bone (ALPL, RUNX2, etc.) or by analysis of the calcic deposits after induction of osteogenesis in 7 to 15 days. This osteogenic induction can be performed by culture of the NSCs in monolayer in the presence of an osteogenic differentiation containing a corticosteroid, such as dexamethasone (0.1-1 μM), a reducing agent such as ascorbic acid 2-phosphate (between 0 and 200 μg/ml) and β-glycerophosphate (0-50 mM). BMP-2 may for example replace dexamethasone.

At the end of the differentiation process, the presence of calcic deposits in the culture dish is shown by colouring with a 1% (weight/volume) Alizarin red solution under microscope. The absence of deposits reveals the absence of osteogenic differentiation potential while the presence of calcic deposits coloured by an intense red show the presence of an osteogenic differentiation.

Alternatively or in addition, a protein and/or transcription study of the specific markers of the bone (ALPL, RUNX2) is carried out.

It should be noted that the MHC-I^{L}/CD90^{H} NSCs have a limited osteogenic differentiation potential compared with the MHC-I^{H}/CD90^{L} MSCs with regard to the quantified markers (ALPL, RUNX2).

The inventors have been able to show that the MCH-I^{L}/CD90^{H} phenotype of the NSCs according to the present disclosure is connected with a low osteogenic differentiation capacity of these cells.

This characteristic thus enables the NSCs to be used in cell therapy. In particular, it is a case of cell therapy for the treatment of arthropathy, since they limit the generation of calcified ectopic tissue within the joint. This property is particularly important for applications such as osteoarthritis, with or without tissue symptoms.

Pharmaceutical Composition and Injectable Solution

Another aspect of the invention relates to a pharmaceutical composition comprising a population of NSCs as described above.

Thus the present invention also relates to a pharmaceutical composition or an injectable solution ready for use, comprising a population of neonatal stromal cells MHC-I^{L}, and optionally CD90^{H} as defined previously, and a pharmaceutically acceptable vehicle.

Typically, the pharmaceutical composition or the injectable solution includes a population of NSCs of 1×10⁶ to 1×10⁸ cells in a volume of 0.1 ml to 15 ml, that is to say a concentration of 5×10⁴ to 1×10⁹ cells/ml. In particular, it comprises from 1×10⁶ to 5×10⁷ cells in a volume of 0.1 ml to 15 ml, that is to say a concentration of 7×10⁴ to 5×10⁷ cells/ml. In particular, it comprises from 2.5×10⁶ to 1×10⁷ cells for a volume of 0.1 ml to 15 ml of composition, that is to say a concentration of 1.5×10⁵ and 1×10⁸ cellules/ml. In particular, it comprises from 1×10⁶ to 1×10⁷ cells in 0.5 to 2 ml, that is to say a concentration of 5×10⁵ to 2×10⁷ cells/ml.

Typically, said injectable solution comprises between 1×10⁶ and 1×10⁸ cells, in particular 1×10⁷ cellules.

“Ready for use” means that the pharmaceutical composition or injectable solution comprising NSCs is ready to be injected into the individual. Putting the cells back into culture before use in the individual is not necessary and the cells do not need to be washed or resuspended in a physiological medium, even when they are formulated with a cryoprotector as described below. The expression “ready for use” means that only one thawing step is necessary when the composition or injectable solution is in frozen form, before injection into the subject.

Since this pharmaceutical composition or injectable solution can be frozen, it can thus be mobilised at any time. This has the advantage of making the treatment available in a short a time as possible while limiting the human action necessary for the efficacy thereof and limiting the risk of contamination inherent in each handling by an operator. Thus it is possible to separate the method for obtaining the pharmaceutical composition from the end clinical use thereof.

Typically, the pharmaceutically acceptable vehicle and/or the packaging makes it possible to maintain the biological properties of the NSCs during a sufficient length of time.

The vehicle may be all types of liquid, gel or solid polymer capable of containing NSCs without damaging the required properties thereof, in particular a saline aqueous solution, serum or culture medium.

The packaging may be any type of receptacle, container or medical device capable of aseptically isolating the pharmaceutical preparations from the external environment and/or from the transport environment and/or from the handler. In particular, the packaging makes it possible to maintain the integrity and the formulation of the pharmaceutical composition and/or to facilitate the distribution/transport of the pharmaceutical composition.

In particular, the pharmaceutically acceptable vehicle may be any solution allowing the freezing and/or thawing of the cells while limiting the biological influence of these processes on the cells such as the causing of cell death, differentiation, causing of cell senescence, osmotic shocks, the causing of membrane porosity, modification of the membrane composition and/or phenotype changes.

In a particular embodiment, the pharmaceutically acceptable vehicle is a solution corresponding to D-PBS (Dulbecco's phosphate-buffered saline), DMEM (Dulbecco's Modified Eagle Medium), MEM (Minimum Essential Media), a solution comprising foetal calf serum (FCS), a solution comprising animal serum, and/or any other isotonic solution.

In a particular embodiment, said solution comprises between 0 and 20% FCS, more particularly between 5 and 20%, even more particularly 10%. In a particular embodiment, said solution is free from product of animal origin.

In a particular embodiment, said pharmaceutically acceptable vehicle is a solution comprising a cryoprotector. Since the whole of the formulation of the solution is compatible with in vivo injection, the solution comprising a cryoprotector can be used as an injectable solution for treatments to which the pharmaceutical composition described in said invention relate.

Cryoprotector means any compounds for fulfilling the cryopreservation function. According to a particular embodiment, said cryoprotector is chosen from glycerol, Dimethylsulfoxide (DMSO), propylene glycol, proteoglycans, trehalose, bovine serum albumin (BSA), gelatine, polyethylene glycol (PEG), polyacrylic acid, poly-L-lysine, ethylene glycol or a combination of any plurality of these cryoprotectors. In one particular embodiment, said solution comprises between 0.5 and 30%, in particular 0.5 and 20%, in particular 2 and 10%, more particularly 5% cryoprotector.

Commercial solutions comprising a cryoprotector that can be used in the pharmaceutical composition are preformulated synthetic cryoprotection solutions of the StemAlpha, CryoStor® CS2, CS5 or CS10 type.

In a particular embodiment, said solution comprises 0.5 to 30% glycerol, 0.5 to 30% DMSO, 0.5 to 30% propylene glycol or 0.5 to 20% poly-L-lysine. In particular, said solution comprising a cryoprotector is a solution comprising 2 to 10% DMSO, more particularly 5% DMSO. In particular, said solution comprising a cryoprotector is a solution comprising 2 to 20% glycerol.

In a particular embodiment, one or more adjuvants can be added, such as ammonium chloride, Ringer lactate or BSA.

In a particular embodiment, said solution comprising a cryoprotector is a DMEM solution comprising FCS, in particular 5 to 20% FCS and more particularly 10%.

In a particular embodiment, said solution comprising a cryoprotector is free from product of animal origin and comprises 1 to 90% DMSO, more particularly 0.5 to 30%, more particularly 2 to 10%, in particular 5%.

In another particular embodiment, said solution comprising a cryoprotector is a DMEM solution comprising FCS, in particular 5 to 20% FCS and more particularly 10%, and from 2 to 20% glycerol.

In a particular embodiment, the pharmaceutical composition or injectable solution is characterised in that it is in frozen form.

The pharmaceutical composition or injectable solution may be frozen with the use of a suitable cryoprotector, capable of guaranteeing the integrity and the formulation of the pharmaceutical composition. The frozen pharmaceutical composition can then be stored at negative temperature between −70° C. and −196° C., and more particularly at temperatures below −70° C. for long-term storage (greater than 12 months). In order to be used, it undergoes thawing.

The inventors showed unexpectedly that the biological properties of the pharmaceutical composition cryopreserved for up to 12 months at −80° C. were maintained (Example E) (FIGS. 8A, 8B, 8C and 8D). Furthermore the therapeutical efficacy of the pharmaceutical composition of NSCs of MHC1^{L}/CD90^{H} phenotype cryopreserved was shown by the treatment of canine osteoarthritis, with an improvement in the mobility of the animal, evaluated by means of a validated questionnaire (LOAD: Liverpool Osteoarthritis in Dogs) (FIG. 9), and/or by a clinical evaluation made by the veterinary surgeon (FIG. 10). In a particular embodiment, the inventors also showed the therapeutic efficacy of the pharmaceutical composition of NSCs of MHC1^{L}/CD90^{H} phenotype cryopreserved for the treatment of thrombocytopenia (FIG. 11). The inventors have also shown the therapeutic efficacy of the pharmaceutical composition of NSCs of MHC1^{L}/CD90^{H} phenotype cryopreserved for the treatment of chronic inflammatory diseases (CIDs) (Example H).

An injectable composition or solution in frozen form “ready to use” according to the invention is thus mobilizable at all times. This has the advantage of making the treatment available in the shortest possible time while limiting the human action necessary for its efficacy and limiting the risk of contamination inherent in each handling by an operator. Thus it is possible to separate the method for obtaining the pharmaceutical composition from the final therapeutic use thereof.

In one embodiment, the present invention relates to a ready-for-use injectable solution comprising a population of MHC-I^{L} NSCs, and optionally CD90^{H}, as described previously, and a cryoprotector as defined previously.

Typically said population of NSCs and said cryoprotector are formulated in a pharmaceutically acceptable vehicle as defined previously.

In a particular embodiment, said ready-for-use injectable solution comprises a unit dose of MHC-I^{L} NSCs, and optionally CD90^{H}, of 1×10⁶ to 1×10⁸ cells, in particular 1×10⁷ cells, and a solution comprising a cryoprotector as defined previously.

In a particular embodiment, said injectable solution comprises a unit dose of 1×10⁶ to 1×10⁸ cells in a volume of 0.1 to 15 ml, more particularly 1×10⁶ to 1×10⁷ cells, typically 1×10⁷ cells, in a volume of 0.5 to 2 ml.

In a particular embodiment, at least 80% in number, in particular at least 85%, more particularly at least 90% of the NSCs are of MHC-I^{L}/CD90^{H} phenotype. In a more particular embodiment, the NSCs are placental NSCs, more particularly of canine origin.

In a particular embodiment, the cryoprotector in said injectable solution is DMSO. In a particular embodiment, the injectable solution is free from any product of animal origin and comprises from 1 to 90% DMSO, more particularly 0.5 to 30%, more particularly 2 to 10%, in particular 5%.

In a particular embodiment, the NSCs may undergo an exogenous stimulation/modification before injection by means of physical, biological and/or chemical effectors. Exogenous stimulation (also referred to as “priming”) means any stimulation/modification of the cells and/or of the microenvironment thereof triggering in them a phenotype change favouring the biological properties thereof in the context of specific therapies. For example, a treatment with cytokines in concentrations of between 1 and 500 ng/ml of IFN-γ, of IL-1R, Il-6 and/or TNF-α makes it possible to significantly increase the expression of molecules exerting an immunomodulator activity. In another example, mechanical stimulations and/or the inducing of a chondrogenic predifferentiation may make it possible to favour the properties of in vitro tissue reconstruction.

Neonatal Stromal Cell

Another aspect of the disclosure relates to a neonatal stromal cell of MHC-I^{L} phenotype, in particular of MHC-I^{L}/CD90^{H} phenotype.

This cell can in addition be characterised structurally by the fact that it does not express one or more of the following surface markers: CD11b, CD14, CD31, CD34, CD45, or HLA-DR also referred to as MHC-II.

Moreover, functionally, it can be characterised in that it has:
an ability to adhere to a plastic support; and/or
a capacity for consecutive cell doubling greater than 20 total doublings, and/or
a potential for chondrogenic differentiation, and/or
an immunomodulator potential; and/or
no or little potential for osteogenic differentiation.

In a particular embodiment, the present disclosure relates to a neonatal stromal cell for therapeutic use thereof in dogs, cats, horses or humans, for example for the treatment of:
a. tissue or joint damage, with or without inflammatory component;
b. degenerative illnesses, in particular osteoarthritis, tendinopathies, tissular fibroses, Alzheimer's, Parkinson's;
c. autoimmune, inflammatory and/or infectious diseases, in particular atopic dermatitis, gingivostomatitis, thrombocytopenia, epidermolysis bullosa, sepsis, chronic inflammatory bowel diseases (CIBD);
d. graft rejection, or
e. tumoral diseases.

Such an NSC cell can be isolated advantageously from the population of NSC cells according to the present disclosure as described above, in accordance with the cloning methods known to a person skilled in the art.

Therapeutic Use

Another aspect of the present invention relates to a population of NSCs, a pharmaceutical composition or an injectable solution as defined previously for therapeutic use thereof.

Typically, it is a case of use in cell therapy. “Cell therapy” means a therapeutic treatment comprising the administration of cells able to induce a beneficial therapeutic effect in the individual. In the context of a regenerative medicine approach, this cell therapy is able to favour directly (cell differentiation) or indirectly (secretion of biological factors, activation or inhibition of cells of the environment) the in vivo regeneration of one or more biological tissues in an individual awaiting such treatment.

In particular, it is a case of a use in the same individual, or in an individual of the same species, or in an individual of a species that is different from the species from which said NSCs come.

When the receiving subject is identical to the individual from which the NSCs come, an autologous therapeutic use is spoken of.

When the receiving subject is an individual of the same species as the species from which the NSCs come, heterologous or allogenic therapeutic use is spoken of.

When the receiving subject is an individual from a species that is different from the species from which the NSCs come, xenogenic therapeutic use is spoken of.

In a particular embodiment, the present invention relates to a population of NSCs as defined previously for xenogenic therapeutic use thereof.

In a particular embodiment, the present disclosure relates to said population of NSCs, of a pharmaceutical composition or an injectable solution as defined previously for therapeutic use thereof in a mammal. In particular, said mammal is a dog, cat, horse or human.

By way of example, the therapeutic use may be the treatment of:
a. tissue or joint damage, with or without inflammatory component;
b. degenerative illnesses, in particular osteoarthritis, tendinopathies, tissular fibroses, Alzheimer's, Parkinson's;
c. autoimmune, inflammatory and/or infectious diseases, in particular atopic dermatitis, gingivostomatitis, thrombocytopenia, epidermolysis bullosa, sepsis, chronic inflammatory bowel diseases (CIBD);
d. graft rejection, or
e. tumoral diseases.

Tissue damage means lesions, loss of normal function and/or degradations caused by an excessive stress on the tissue, normal stress on a pathological tissue or all tissues requiring a tissue reconstruction/healing: myocardial infarction, renal damage, liver damage, burn, skin lesions, fractures, respiratory diseases, osteoarticular diseases such as osteochondritis dissecans.

Degenerative illness means all types of illness where the homeostatic balance is disturbed in favour of an exacerbated tissue catabolism or an excessive induction of tissue anabolism, such as for example: osteoarthritis, tendinopathies, tissue fibroses, Alzheimer's or Parkinson's.

NSCs can also be used in the treatment of physiological diseases or disturbances related to an undesired immune response, that is to say all types of diseases where the immune system interferes with the normal/physiological functioning of the tissue or with a treatment aimed at treating an immune disease and/or resorbing he normal/physiological functioning of a tissue, such as for example atopic dermatitis or gingivostomatitis.

They can be used in the treatment of autoimmune and inflammatory diseases such as reaction of the graft against the host (graft versus host disease or GvHD, tissue or organ graft, autoimmune diseases such as multiple sclerosis, tissue inflammation, allergy, asthma, allergenic bronchitis, chronic bronchitis such as chronic obstructive pulmonary bronchopathy (COPB), chronic inflammatory bowel disease, renal insufficiency, thrombocytopenia, lupus erythematosus, rheumatoid arthritis and epidermolysis bullosa.

“Chronic inflammatory bowel diseases (CIBD)” means chronic inflammations of the mucosa of the small intestine, of the colon and of the anoperineal region that are idiopathic, such as duodenal enteritis in dogs and cats, and more particular inflammation such as Crohn's disease and haemorrhagic rectocolitis in humans. These illnesses are characterised by gastrointestinal problems and chronic problems associated with inflammatory infiltration of the mucosa. They are usually diagnosed when there is vomiting and chronic diarrhoea in animals and humans. CIBDs are distinct from enteropathies responding to a change in food and diarrhoeas responding to antibiotics. By definition, they respond to immunosuppressors and not a specific food or to antibiotics. The clinical signs such as vomiting, diarrhoea, weight loss and loss of appetite are due to cells infiltrating the mucosa, to inflammation mediators, to a malfunctioning of the enterocytes associated with the inflammation and to disturbance to the motility of the intestine.

The NSCs according to the invention can also be used in the treatment of diseases in which chronic inflammation related or not to an immune disorder causes tissue degenerescence or malfunctioning of the function of an organ or of a tissue such as osteoarthritis or tendinitis. Moreover, said population of NSCs, pharmaceutical composition or injectable solution can be used in the treatment of infectious diseases associated or not with the previous components. Infectious diseases means diseases involving a contamination by pathogens such as microorganisms such as protozoa, bacteria and/or viruses. These diseases with an infectious component may be of a localised or systemic nature. The use of the NSCs can in particular be prescribed in the particular context of resistance to antibiotics of the pathogens involved and in the context of a generalised inflammatory response associated with a serious infection, such as sepsis.

The therapeutic context can extend to diseases having several pathological aspects such as osteoarthritis with a degenerative aspect and an inflammatory aspect.

Said population of NSCs, pharmaceutical composition or injectable solution can also be used in the treatment of diseases of a tumoral nature with or not a metastatic component.

The therapeutic context can also extend to the use of the NSCs combined with other types of therapy such as for example: laser; shockwaves; platelet-rich plasma (PRP); hyaluronic acid; non-steroidal anti-inflammatories.

In a particular embodiment, the present disclosure relates to a population of NSCs for use thereof in tissue engineering. “Tissue engineering” means all the biotechnology techniques using cells and biomaterials (of biological or synthetic origin) for generating tissue substitutes in vivo/ex vivo for an in vivo implantation and for being used a tissue model in the laboratory (e.g. skin, bone or cartilage reconstruction).

In a particular embodiment, the present disclosure relates to a population of NSCs, a pharmaceutical composition or an injectable solution as defined previously for use thereof in the treatment of indications of the musculoskeletal system such as osteoarthritis in mammals, more particularly in dogs, cats or horses, preferably in dogs.

In another particular embodiment, the present disclosure relates to said population of NSCs, pharmaceutical composition or injectable solution as defined previously for use thereof in the treatment of thrombocytopenia in mammals, more particularly in dogs, cats or horses, preferably in dogs.

In a particular embodiment, the present disclosure relates to said population of NSCs, a pharmaceutical composition or injectable solution as defined previously for use thereof in the treatment of chronic inflammatory bowel diseases, such as duodenal enteritis in mammals, more particularly in dogs, cats or horses, preferably in dogs.

In a particular embodiment, the present disclosure relates to said population of NSCs, pharmaceutical composition or injectable solution as defined previously for use thereof in the treatment of chronic inflammatory bowel diseases, such as Crohn's disease in humans.

Treatment Method

According to another aspect, the present invention relates to a method for treating:
a. tissue or joint damage, with or without inflammatory component;
b. degenerative illnesses, in particular osteoarthritis, tendinopathies, tissular fibroses, Alzheimer's, Parkinson's;
c. autoimmune, inflammatory and/or infectious diseases, in particular atopic dermatitis, gingivostomatitis, thrombocytopenia, epidermolysis bullosa, sepsis, chronic inflammatory bowel diseases (CIBDs);
d. graft rejection, or
e. tumoral diseases.

comprising the administration of said population of NSCs or of a pharmaceutical composition or of an injectable solution as defined previously in the subject to be treated.

In particular, the present invention relates to a method for treating indications of the musculoskeletal system such as osteoarthritis in mammals, in particular in dogs, cats or horses, preferable in dogs, comprising the administration of said population of NSCs or of a pharmaceutical composition or an injectable solution as defined previously in the subject to be treated.

In particular, the present invention relates to a method for treating the treatment of thrombocytopenia in mammals, more particularly in dogs, cats or horses, preferably in dogs, comprising the administration of said population of NSCs or of a pharmaceutical composition or an injectable solution as defined previously in the subject to be treated.

In particular, the present invention relates to a method for treating the treatment of chronic inflammatory bowel diseases in mammals, more particularly in dogs, cats or horses, preferably in dogs, comprising the administration of said population of NSCs or of a pharmaceutical composition or an injectable solution as defined previously in the subject to be treated.

Administration Method

The population of NSCs, a pharmaceutical composition or an injectable solution as defined previously can be administered locally or intravenously (IV) or more generally parenterally.

Typically, a local administration may be an administration by intra-articular injection, for example in the case of treatment of indications of the musculoskeletal system such as osteoarthritis, at each joint to be treated.

IV administration means an administration of the NSCs directly into the venous system of the subject using a catheter or a needle or for example via a pouch of perfusion solute or for example through the tube of the perfuser. Typically, an administration intravenously is performed in the case of the treatment of thrombocytopenia, osteoarthritis or the treatment of a chronic inflammatory bowel disease such as duodenal enteritis or Crohn's disease.

In a particular embodiment, 1×10⁶ to 1×10⁸ NSCs, in particular 2.5×10⁶ to 1×10⁷ NSCs, more particularly 1×10⁷ NSCs, are administered by intra-articular route, at each joint to be treated, for the treatment of indications of the musculoskeletal system such as osteoarthritis.

In a particular embodiment, 1×10⁶ to 10×10⁸ NSCs, in particular 1×10⁷ NSCs, are administered intravenously for the treatment of inflammatory diseases involving a problem with the immune system, such as thrombocytopenia.

In a particular embodiment, 1×10⁶ to 5×10⁶ NSC/kg are administered intravenously for treating inflammatory diseases involving a problem with the immune system, such as thrombocytopenia.

In a particular embodiment, 1×10⁶ to 10×10⁸ NSCs, in particular 1×10⁷ NSCs, are administered intravenously for treating a chronic inflammatory bowel disease.

The administration method is obviously adapted according to the subject and the pathology to be treated. The exact number of cells to be administered depends on various factors, and in particular the age, the weight and the sex of the subject to be treated, the pathology and the extent or severity of the pathology to be treated.

Method for Obtaining

The present disclosure also relates to a method in vitro for obtaining a pharmaceutical composition of neonatal stromal cells coming from neonatal tissues (FIG. 16 and FIG. 17), said pharmaceutical composition comprising as active substance a population of neonatal stromal cells comprising NSC of phenotype MHC-I^{L}, and optionally of phenotype CD90^{H}, said method comprising:
a. supplying one or more neonatal biological samples comprising NSC, the biological sample or samples having been obtained beforehand from one or more individuals,
b. isolating the population of NSC present in the biological sample or samples,
c. optionally, at least one amplification step ex vivo of the NSC obtained in step b.,
d. optionally, the cryopreservation of the population of NSC obtained in step b. or c.,
e. optionally, a stimulation by physical, biological and/or chemical effector of the population of NSC obtained in step b., c. or d.,
f. characterising the presence of NSC of phenotype MHC-I^{L}, and optionally of a phenotype CD90^{H}, among at least 80% of the population of NSC, after isolation in step b. and/or after the amplification step ex vivo in step c. and/or after cryopreservation of the NSC in step d.,
g. putting into suspension of the population of NSC comprising at least 80% of NSC of phenotype MHC-I^{L}, and optionally of phenotype CD90^{H} in a pharmaceutically acceptable suspension medium.

In an embodiment, the neonatal biological sample or samples supplied in step a., come from a sample of neonatal tissue, in particular from one or more placentas and/or from one or more umbilical cords, or from one or more amniotic membranes, or from a sample of neonatal fluid such as for example the amniotic fluid from one or more amniotic sacs and in particular the blood from one or more umbilical cords. In a particular embodiment, the neonatal biological sample is a placenta.

This sample can be taken as explained hereinabove in the application.

In particular, the neonatal biological sample or samples supplied in step a) of the method come from mammals, and more particularly from dogs, cats, horses or Man. In a particular embodiment, the neonatal biological sample is a sample coming from a dog, also referred to as canine sample.

In a particular embodiment, the neonatal biological sample or samples supplied come from the same individual, from one or more individuals or from a mammal model different from the one for which the pharmaceutical composition is intended.

“Isolation” means the operation consisting of extracting via an enzymatic and/or mechanical process, the cells contained in a tissue and the extracellular matrix thereof.

The isolation of the NSC is carried out from a neonatal tissue, for example, by dissection and enzymatic digestion of the tissue, then by centrifugation and recovery of the cell button containing NSC. Alternatively, it is carried out from a sample of umbilical cord blood. The blood cells can then be separated over a density gradient, in particular by using Ficoll. The cell ring formed at the interphase between the diluted plasma and the Ficoll is recovered, and the cells are washed and centrifuged, then the cell button containing NSC is recovered. The cells are in general counted and seeded at a density comprised between 10⁵ and 5·10⁵ cells/cm2. The total number of cells recovered after centrifugation can be comprised for example between 0.1·10⁶ and 500·10⁶ cellules, more precisely in dogs between 0.1·10⁶ and 10·10⁶, more precisely in horses 100·10⁶ and 500·10⁶.

Following the isolation of the cells containing a fraction of NSC, a step of amplification of the NSC can be carried out by adhesion to the plastic.

In order to obtain more substantial quantities of NSC for the purpose of carrying out different pharmaceutical preparations, the isolated NSC can undergo an amplification step in the laboratory.

“Amplification step” means any step that allows for a proliferation of the NSC on a plastic or polymer support.

This phase must be capable of favouring the presence of the NSC to the detriment of other cell types that do not meet the characteristics of the NSC. It must also ensure an optimum proliferation of the cells while still limiting the phenomena of dedifferentiation, of differentiation and/or of senescence. This step involves conditions in a controlled atmosphere such as those skilled in the art are able to establish such as for example with 90% humidity and including 5% CO₂. The amplification temperature must be constant and comprised between 35-40° C., more precisely between 37-39° C. Along the culture mediums, non-exhaustively, it is possible to mention the mediums of the Alpha-MEM, DMEM, RPMI, IMDM, Opti-MEM, EGM, EGM-2 type, synthetic mediums adapted to the culture of MSC devoid of endotoxin and/or of serum, synthetic mediums adapted to good manufacturing practices, supplemented or not with foetal bovine serum (FBS) from 0.1% to 20%, platelet lysate, insulin-transferrin-selenium, defined commercial supplements and/or other growth factors and/or molecules that favour the proliferation of NSC while still limiting the senescence thereof such as FGF, EGF, VEGF, dexamethasone and/or A2P.

This amplification phase can be carried out on different supports once the NSC population is obtained following the isolation step. These different supports can be of a 2D or 3D nature.

“Amplification sur support 2D” any methods of cell culture allowing for an amplification of the NSC on single-layer support and correctly developed by those skilled in the art. In particular embodiments the cells can be cultivated in plastic culture dishes treated or not to favour cell adhesion, of the flask type, with one or more stages and/or of the multi-layer type with or without continuous perfusion, with or without optimisation of the air flow.

“Amplification on 3D support” means any techniques known by those skilled in the art using biomaterials, microcarriers and/or polymers able to ensure an amplification of the NSC in a bioreactor and correctly developed by those skilled in the art. In particular embodiments, the NSC can be amplified in bioreactors under agitation, axial and/or tilting, under wave agitation, under rotating bed agitation, in static and/or infused bioreactors. The biomaterials and/or microcarriers can be of several natures and according to particular embodiments can be of sizes comprised between 100-500 μm in diameter, have a porosity of a different nature, have a treated surface, negatively or positively charged or not, include growth factors or recombinant proteins of the integrin and/or extracellular matrix type or any other biological/chemical molecules that favour cell adhesion and/or cell proliferation.

As the NSC are adherent cells, in order to ensure the amplification step, a cell passage of the NSC can be necessary and carried out by a method correctly developed by those skilled in the art. A cell passage (P) corresponds to the detaching of cells from their support when they arrive at confluence (cell layer), to put them back into culture on a new support. Typically, the detaching of cells can be carried out under the effect of mechanical action, enzymes and/or inhibitors such as, non-exhaustively, trypsin, EDTA and/or recombinant or animal accutase. It is also possible to carry out these cell passages through the use of biomaterials/microcarriers that can be dissolved according to a method developed by those skilled in the art.

In a particular embodiment, for the amplification phase, the cells are for example treated with trypsin-EDTA, then taken in an amplification medium and centrifuged. After taking in the amplification medium, they are put back into culture at a rate of 1,000 to 5,000 cells/cm² or 3D equivalent in amplification medium with or without monitored follow-up of the microenvironment and/or culture atmosphere.

In a particular embodiment, the amplification step can comprise several cell passages.

During this amplification step, the NSC multiply via cell doubling. Thus, the amplification step can also be defined in terms of cell doubling.

In a particular embodiment, said method comprises an amplification step wherein the NSC population according to the invention undergoes 2 to 25 cell doublings, more particularly 5 to 15 cell doublings.

At the end of the isolation from neonatal tissues, the cells can be cryopreserved in seed units.

For this, the cells are centrifuged then taken in a freezing medium. The freezing medium can either be a culture medium such as for example DMEM enriched with 5-50% FBS (vol:vol) and 5-10% (vol:vol) of DMSO or a commercial cryopreservation medium, containing or not a fraction of DMSO. The freezing of the cells is carried out for example in controlled falling temperature conditions (−1° C./min to a temperature of −80° C.), by using for example a CoolCell® Cell Freezing Container (BioCision) or a programmable freezer of the Digitcool (Cryobiosystem) type. The NSC thus frozen can be stored in particular at temperatures below −70° C. for long-term storage (greater than 12 months).

The characterisation step of the presence of a phenotype MHC-I^{L}, and optionally of phenotype CD90^{H}, is carried out according to the methods described hereinabove of single marking or double marking. This step is carried out on a sample of the population of NSC coming from step b., c. and/or d., to characterise said population. The purpose of this step is to select a population of NSC comprising at least 80% of its cell population of phenotype MHC-I^{L}, and optionally of phenotype CD90^{H}. The populations of NSC that meet this criterion are selected and can be put into suspension in a pharmaceutically acceptable suspension medium, such as for example a buffered saline solution, an injectable sterile solution containing 10-100 USP of heparin sodium, a commercial cryopreservation medium used as an excipient.

In a particular embodiment, the characterisation step consists of characterising NSC MHC-I^{L} and CD90^{H}, and is carried out via double marking of the MHC-I and CD90 markers in flow cytometry, such as described hereinabove. The characterisation step of the presence of a phenotype MHC-I^{L}/CD90^{H} corresponds to an evaluation of rMFI MHC^{L} less than 20, more particularly less than 15, more particularly less than 10 and according to an evaluation of rMFI CD90 greater than 15 and more particularly greater than 20, among at least 80% of the NSC of the population of NSC.

In a particular embodiment, said method comprises an amplification step c., said amplification step comprising 1 to 5 cell passages, more particularly comprising 2 or 3 cell passages. In this embodiment, characterising the population of NSC isolated in step b. is therefore carried out after an amplification step comprising 1 to 5 cell passages (step c.), more particularly comprising 2 or 3 cell passages.

In an embodiment, said method comprises an amplification step c., said amplification step corresponds to a doubling of the NSC population isolated in step b. from 2 to 25 cell doublings, in particular 5 to 15 cell doublings. In this embodiment, characterising the population of NSC isolated in step b. is therefore carried out after an amplification step corresponding to a doubling of the NSC population isolated in step b. from 2 to 25 cell doublings, in particular 5 to 15 cell doublings.

In an embodiment, said amplification step can be characterised in terms of passages and in terms of cell doublings such as defined hereinabove.

The inventors have observed that between the passage 1 (P1) and the passage 5 (P5), in particular between the passage 2 (P2) and the passage 3 (P3), a substantial phenotypic change takes place. Indeed, they have surprisingly discovered that the phenotypic profiles of the different populations of NSC are similar between P0-P1 and are mainly of the intermediate MHC-I^{H} and CD90 type. On the other hand between P1 and P5, more particularly between P2 and P3, an individualisation of the sub-populations of NSC of a different phenotype can be observed. “Individualisation” means that two sub-populations MHC-I^{L}/CD90^{H} and MHC-I^{H}/CD90^{L} can be observed within the same population of NSC, during cell amplification. Indeed, during the amplification in vitro between P1 and P5 and more particularly between P2 and P3, the populations of NSC pass through phase of individualisation where the two sub-populations MHC-I^{H}/CD90^{L} and MHC-I^{L}/CD90^{H} can be identified easily, in case of a heterogenous population.

In an alternative embodiment, it is possible to combine the notion of passage with the number of doublings: the individualisation phase can then be considered between 2 and 25 doublings, in particular 5 to 15 doublings.

Typically, the characterisation of the marker expression is carried out by the double marking method described hereinabove.

In the case where this individualisation can be observed, an analysis matrix that is specific to the isolated population of NSC of phenotype MHC-I^{L}/CD90^{H}, can be determined in order to be used for the analysis of the sub-populations that it contains and/or therapeutic units and banks produced from this same population of NSC (FIG. 16 and FIG. 18).

“Analysis matrix” means an analysis window determined for the analysis in flow cytometry. The purpose of this matrix is to define the stage of individualisation and/or the phenotype of a given population of NSC, during the steps of amplification and/or after thawing and/or during the method of manufacturing the pharmaceutical preparation.

Using this matrix as a characterisation tool entails at least an analysis at the first passage, in particular at least at the second passage in order to correctly assess the proportion of the different sub-populations of NSC during the method of manufacturing.

In a particular embodiment, this individualisation phase and the establishing of an analysis matrix can be used so as to adjust the determination of thresholds described hereinabove, for the characterisation of marker expression. This phenomenon can be used so as to determine the rMFI of the MHC-I and of the CD90 for each one of the sub-populations within the initial population itself (FIG. 3). The analysis, also, of a sufficiently large number of heterogenous populations of NSC can make it possible to establish thresholds with more precision for the determination of the phenotype MHC-I^{L}/CD90^{H} during the method of fabrication of the invention.

In a particular embodiment, the method for obtaining the pharmaceutical composition of NSC comprises a step of enrichment of the population of NSC in NSC of phenotype MHC-I^{L}, and optionally of phenotype CD90^{H}, which can correspond to a depletion step of the NSC cells of phenotype MHC-I^{L}, and optionally of phenotype CD90^{H}. This enrichment step by cell depletion can be carried out by cell sorting methods of column chromatography, through the use of magnetic beads coupled to antibodies of interest, or by flow cytometry coupled to a cell sorter.

In a particular embodiment, the method for obtaining the pharmaceutical composition of NSC can also comprise a step of inhibiting the MHC-I, for example by modification in vitro of the phenotype of the cells (extinction of a gene) or by masking receptors for example by using an antibody.

At the end of the method, the preparation of NSC can then be amplified in vitro, cryopreserved or administered to an individual afflicted with one of the pathologies mentioned hereinabove.

During the method, it is possible to proceed with a step of cryopreservation, after the putting into suspension of the NSC in a pharmaceutically acceptable suspension medium comprising a cryoprotectant. “Cryopreservation” means the step of storing frozen cells for a period ranging from 1 day to 5 years and more. The cell bank is conditioned in such a way as to guarantee the integrity and the biological properties of the cell populations.

The cryopreservation step is preceded by a step of freezing that corresponds to the falling in temperature of the cell suspension to its storage temperature.

In a particular embodiment, this freezing step corresponds to a progressive drop in the temperature of the cell suspension (−1° C./minute) to reach the storage temperature ranging from −70° C. to −196° C. In an alternative embodiment, the cells can be frozen at a freezing speed comprised between −0.3° C./minute and −99° C./minute. The same freezing protocol can comprise one or more different freezing speeds such as in the case of a gradual rising in the freezing speed. In an alternative embodiment, the cells are frozen directly without temperature control in a storage enclosure of which the temperature is comprised between −70° C. and 196° C. The final storage can be in the liquid phase (of the liquid nitrogen type) or gas phase (of the −140° C., −80° C. enclosure type or storage in nitrogen in the gas phase).

In order to make the cryopreserved cells available for therapeutic administration to the subject, a step of thawing is carried out following the step of freezing, in the case where the NSC used for the administration are in the form of bank units. This thawing is carried out in such a way as to pass from the stage of frozen cells to the stage of thawed cells during an embodiment that limits cell death via desiccation, mechanical lesion of the plasma membrane, osmotic shock. In a particular embodiment, the NSC units are heated by manual friction for less than 10 min. In another particular embodiment, the NSC units are placed in a liquid or dry water bath, adjusted to a temperature comprised between 30 and 40° C. for at least 10 min, in particular at 37° C. for 3 to 5 min. In another particular embodiment, the NSC units are placed in an automatic thawing apparatus. In another particular embodiment, the NSC units are thawed at ambient temperature for less than 10 min if the cryoprotectant used so allows.

Population of NSC Obtained by the Method

The present invention also relates to a population of NSC such as defined hereinabove, obtained by a method comprising:
a. supplying one or more neonatal biological samples comprising NSC, the biological sample or samples having been obtained beforehand from one or more individuals,
b. isolating the population of NSC present in the biological sample or samples,
c. an amplification step ex vivo of the NSC obtained in step b.,
d. optionally, the cryopreservation of the population of NSC obtained in step b. or c.,
e. optionally, a stimulation by physical, biological and/or chemical effector of the population of NSC obtained in step b., c. or d.,
f. characterising the presence of NSC of phenotype MHC-I^{L}, and optionally of a phenotype CD90^{H}, among at least 80% of the population of NSC, after isolation in step b. and/or after the amplification step ex vivo in step c. and/or after cryopreservation of the NSC in step d.,

In a particular embodiment, said amplification step comprises 1 to 5 cell passages, more particularly comprising 2 or 3 cell passages.

In a particular embodiment, said amplification step correspond to a doubling of the population of NSC isolated in step b. from 2 to 25 cell doublings, in particular 5 to 15 cell doublings.

After characterisation, said population of NSC can be formulated as a pharmaceutical composition or injectable solution, such as described hereinabove.

### FIGURES

FIG. 1: Configuration and cytometric analysis of NSC.

(A) Definition of an analysis window based on the analysis parameters FSC-A/SSC-A, then of an analysis window based on the analysis parameters FSC-H/FSC-A. (B) A 2D representation (bar chart with 2 parameters) is used based on the analysis of the fluorescence coming from the fluorochrome used (FL:2 for PE) and of the autofluorescence of the cells by using a fluorescence channel with an excitation/emission spectrum that is not used in the analysis (FL-4). An analysis window is defined on the samples marked by the isotype, for a negativity threshold with a tolerance of about 0.5%. (C) Analysis of the marker MHC-I. (D) Analysis of the marker CD90. (E) A representation by superposition of the bar charts is carried out by retaining the same parameters.

FIG. 2: Revealing the differences in MHC-I expression in NSC.

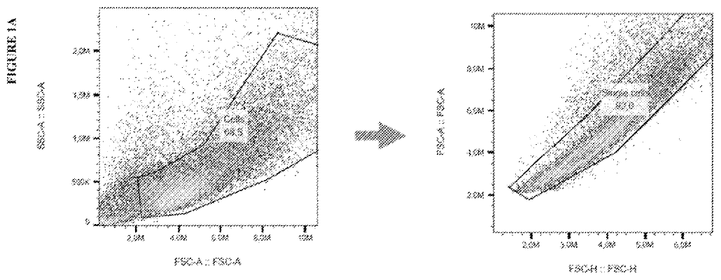

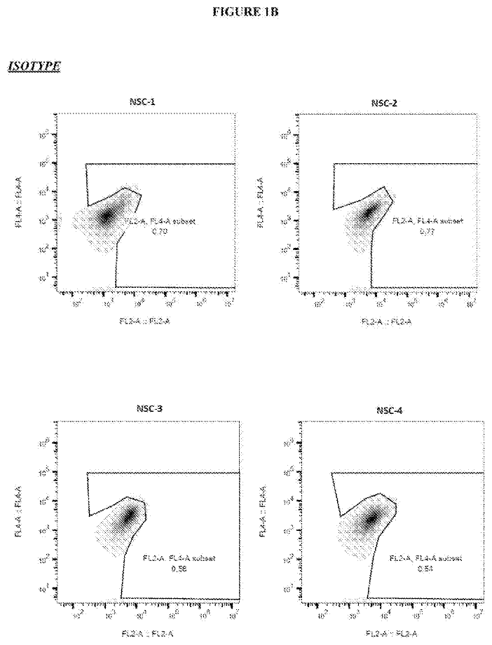

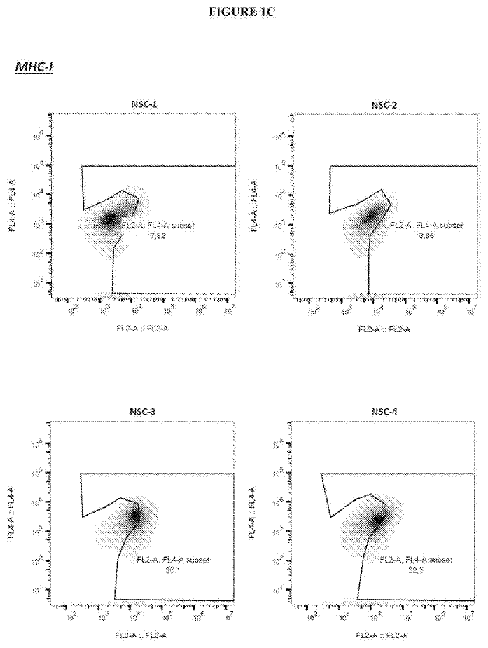

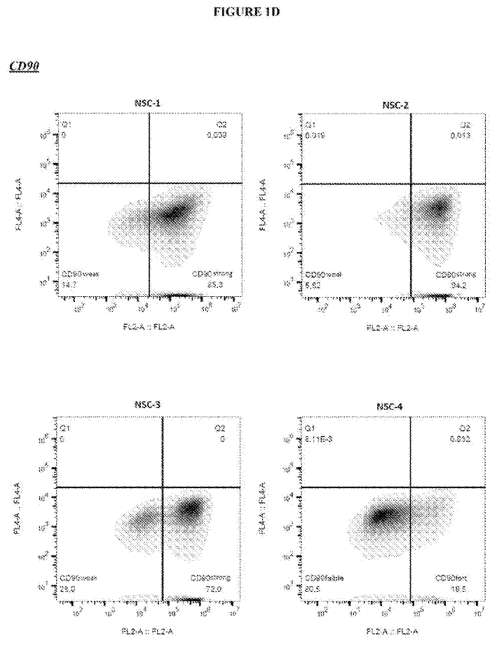

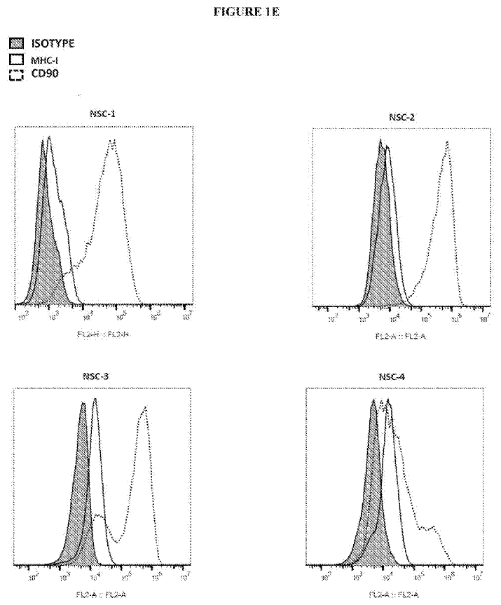

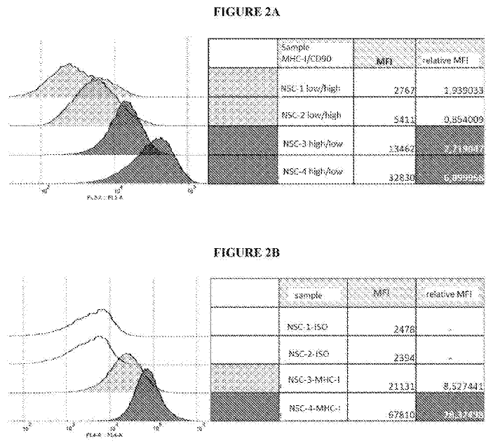

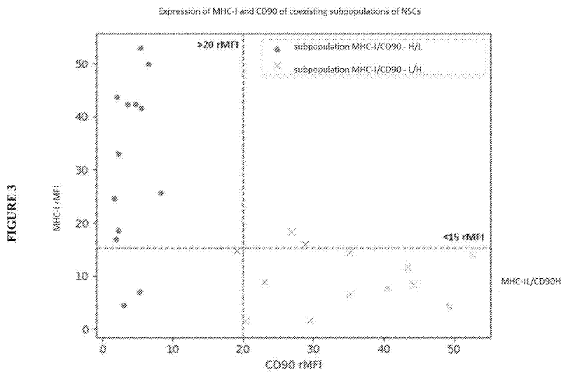

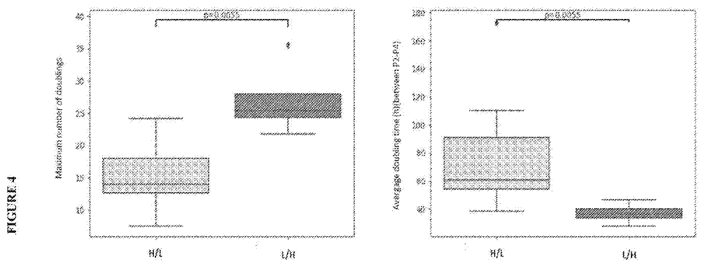

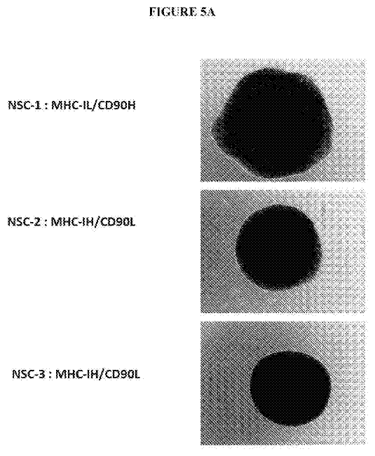

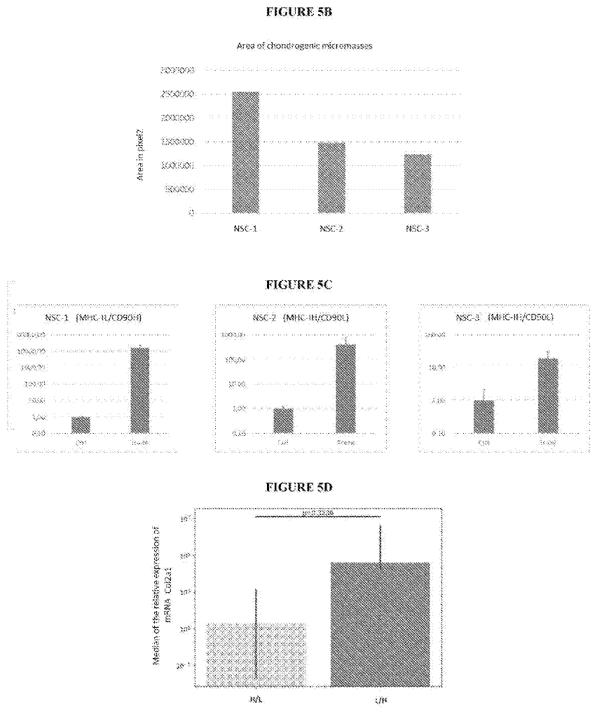

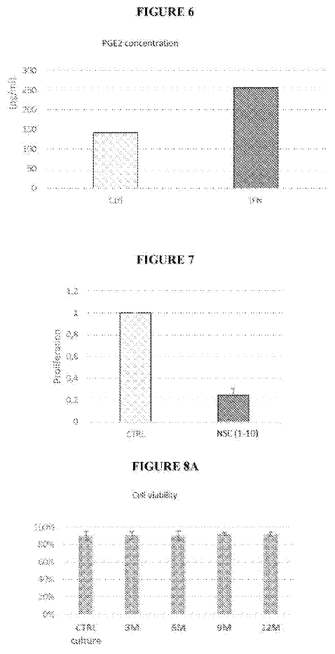

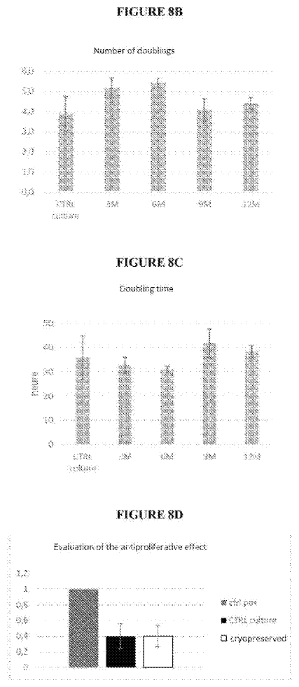

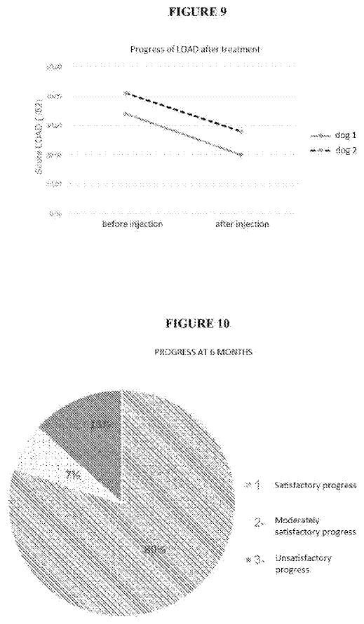

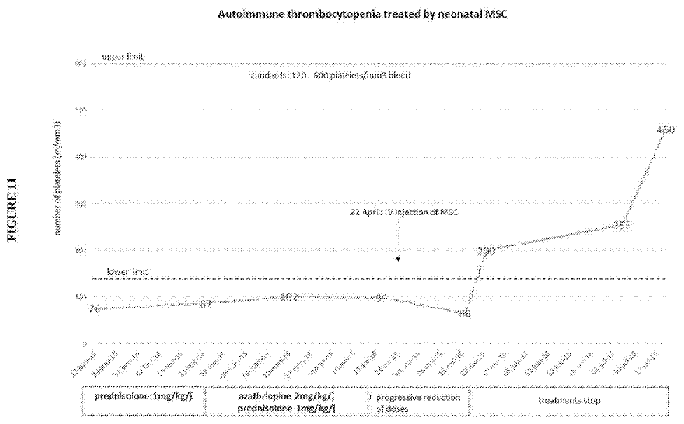

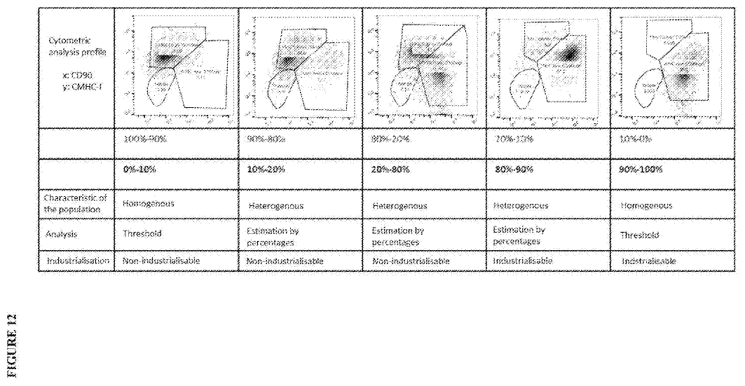

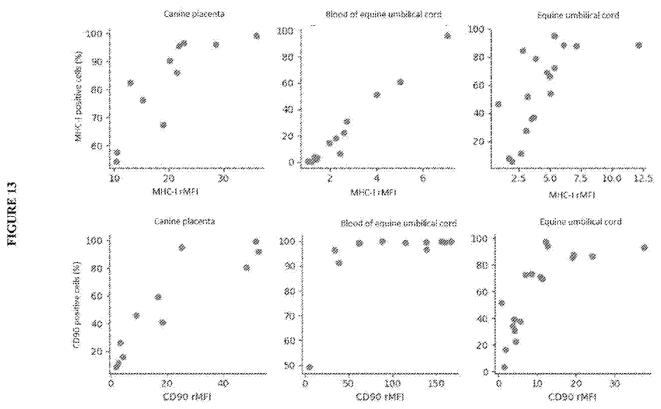

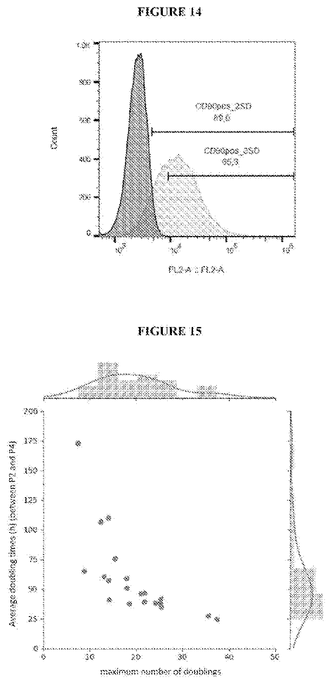

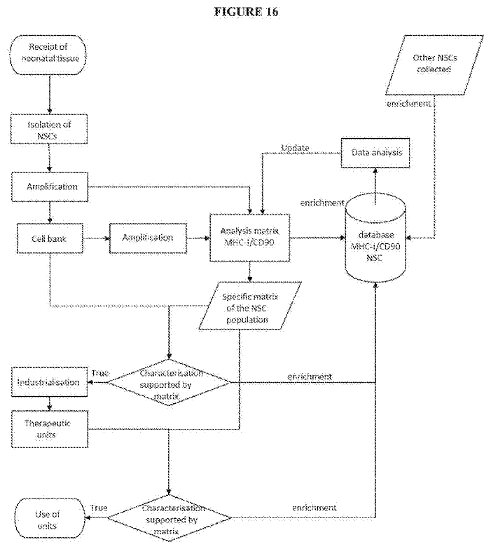

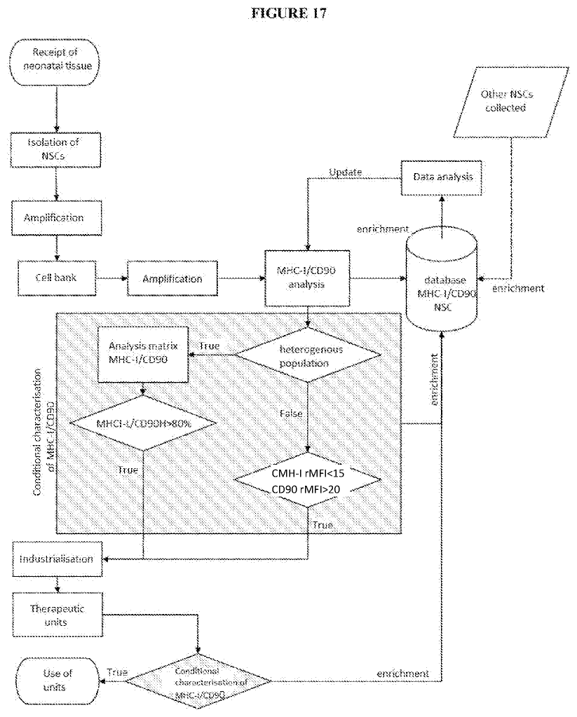

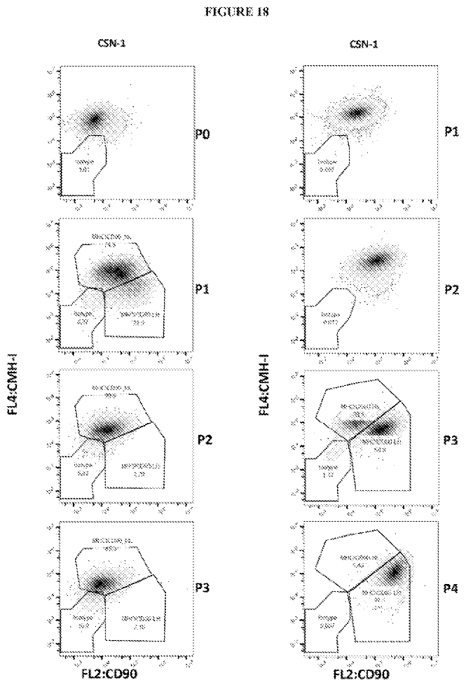

Based on the configuration and the analysis shown in FIG. 1, several samples of NSC are analysed. The analysis of the MHC-I expression indicates variable expressions of this marker between the different samples. The analytical method proposed consists of normalising the mean fluorescence values (MFI; Mean Fluorescence Intensity) of the cells marked with the antibody of interest (here MFI FL2_{(MHC-I-PE)}) with the MFI of the cells marked with the isotype coupled to the same fluorochrome (here MFI FL2_{(isotype-PE)}). A ratio is thus calculated MFI FL2_{(MHC-I-PE)}/MFI FL2_{(isotype-PE)}. (A) Analysis of the marker MHC-I. The acceptability threshold for defining the negative MHC-I cells was set to 2.5. (B) The parameters and the analyses are carried out in accordance with the description of FIG. 1 with the difference that a secondary antibody coupled to a fluorochrome of the allophycocyanin (APC) type is used. The cells marked by an isotype (NSC-ISO) and by the MHC-I (NSC-MHC-I) are presented. In the same way, the analytical method consists of normalising the mean fluorescence values (MFI; Mean Fluorescence Intensity) of the cells marked with the antibody of interest (here MFI FL4_{(MHC-I-APC)}) with the MFI of the cells marked with the isotype coupled to the same fluorochrome (here MFI FL4_{(isotype-APC)}). A ratio is thus calculated MFI FL4_{(MHC-I-APC)}/MFI FL4_{(isotype-APC)}. The acceptability threshold for defining the negative MHC-I cells was set to 10.

FIG. 3: Comparison of the expressions of markers MHC-I and CD90 between the two sub-populations present in heterogenous (mixed) populations of NSC.

NSC are isolated from canine placenta and analysed in flow cytometry between P1-P4. When a mixed population is identified, the MHC-I and CD90 expressions are evaluated in each one of the respective sub-populations MHC-I^{H}/CD90^{L} (MHC-I/CD90-HL) and MHC-I^{L}/CD90^{H} (MHC-I/CD90-LH). The results are presented in the form of relative MFI (rMFI). The analysis makes it possible to establish with precision thresholds of MHC-I and CD90 expressions for the population of interest MHC-I^{L}/CD90^{H} (n=13).

FIG. 4: Proliferation potential difference between the NSC of type MHC-I^{L}/CD90^{H} and MHC-I^{H}/CD90^{L}.

NSC are isolated from canine placenta and analysed in flow cytometry over several passages between P2 and P7. An analysis matrix shown in FIG. 18 is determined for each population of isolated NSC that had the two sub-populations MHC-I^{L}/CD90^{H} and MHC-I^{H}/CD90^{L} during culture. The NSC are characterised as MHC-I^{L}/CD90^{H} when the analysis median of the percentages of the sub-population MHC-I^{L}/CD90^{H} (passages between P2-P7) represents at least 75% of the total population. The results of the maximum number of doublings and of the average doubling time are presented in the form of a boxplot (MHC-I^{L}/CD90^{H} n=4; MHC-I^{H}/CD90^{L} n=11). The probability p of HO rejection is determined by Mann-Whitney U-test.

FIG. 5: Difference in chondrogenic potentials between the NSC of type MHC-I^{L}/CD90^{H} and MHC-I^{H}/CD90^{L}.

A population NSC-1 of phenotype MHC-I^{L}/CD90^{H} and two populations NSC-2 and NSC-3 of phenotype MHC-I^{H}/CD90^{L} were studied after 7 days of chondrogenic differentiation. (A) Observation of the micromasses with a photon microscope (magnification ×4). (B) Bar graph representing the surface of the micromasses analysed under ImageJ and expressed in pixels² according to the cell population. (C) Expression of the gene Col2a1 in the micromasses obtained for each cell population after 7 days of chondrogenic differentiation (treated) in relation to this same cell population that did not undergo chondrogenic differentiation (Ctrl): The levels of expression are presented as relative expression of mRNA Col2a1. (D) NSC are isolated from canine placenta and analysed in flow cytometry over several passages between P2 and P7. An analysis matrix shown in FIG. 18 is determined for each population of isolated NSC having presented the two sub-populations MHC-I^{L}/CD90^{H} and MHC-I^{H}/CD90^{L} during culture. The NSC are characterised as MHC-I^{L}/CD90^{H} when the analysis median of the percentages of the sub-population MHC-I^{L}/CD90^{H} (passages between P2-P7) represents at least 75% of the total population. The results of the chondrogenic potential determined such as described in the invention are presented (MHC-I^{L}/CD90^{H} n=3; MHC-I^{H}/CD90^{L} n=5). The probability p of HO rejection is determined by Mann-Whitney U-test.

FIG. 6: Dosage of PGE2 in the culture supernatants of the NSC MHC-I⁻ (2 independent lines) after 3 days of culture in basal condition (CTRL) or after 3 days of treatment with 5 ng/ml of interferon gamma (IFN)

FIG. 7: Evaluation of the antiproliferative effect of the NSC on the T lymphocytes in vitro This figure shows the anti-proliferative effect of the NSC after 4 days of co-culture of NSC with PBMC (ratio 1:10) in the presence of a mitogenic agent (mitomycin). The control (CTRL) is a culture of PBMCs in the same conditions in the presence of a mitogenic agent but without NSC. The analyse of the lymphocyte proliferation is determined by evaluating the signal of a fluorescent marker (Celltrace) in the population marked with an anti-CD3 antibody (specific marker of T lymphocytes) coupled with a fluorochrome. A proliferation index (PI) is calculated for each experimental condition using the Modfit® analysis software. The PI in the presence of NSC is normalised in relation to the PI in control conditions, set to 1.

FIG. 8: Evaluation of the biological properties of the post-thawing cryopreserved pharmaceutical composition

(A): Evaluation of the cell viability of the NSC before cryopreservation (CTRL culture; n=3) and after thawing of the cryopreserved cells kept 3, 6, 9, and 12 months at −80° C. (3M, 6M, 9M, 12M; n=3 for each condition),

(B-C): The proliferative activity of the NSC coming from a sub-culture (CTRL culture; n=6) or coming from cryopreserved units for 3, 6, 9, 12 months (3M, 6M, 9M, 12M; n=3 for each condition) was evaluated by seeding 225,000 cells in a T75 bottle, kept in culture in an incubator for 7 days. The amplification medium is renewed once during the culture. The number and the doubling time are calculated according to the formula described in the example. The results do not show any significant difference in the number and in the double time between the NSC kept in culture and the cryopreserved NSC, up to 12 months of storage at −80° C. (D): The analysis of lymphocyte proliferation in co-culture with NSC in culture (n=3) or cryopreserved NSC (n=6) does not reveal any significant difference in the antiproliferative effect of the NSC.

FIG. 9: Evaluation of the mobility of dogs before and after administration of cryopreserved NSC based on the LOAD questionnaire.

Two arthrosic dogs are treated with 1·10⁷ of NSC by articulations of phenotype MHC-I^{L}/CD90^{H} cryopreserved and thawed at ambient temperature. The clinical change in the subjects is carried out by analysis of the LOAD score after treatment such as described in example E. The two subjects presented showed a decrease in the LOAD score following the treatment by the cells.

FIG. 10

15 arthrosic dogs are treated with 1·10⁷ of NSC by articulation of phenotype MHC-I^{L}/CD90^{H} cryopreserved and thawed at ambient temperature. The clinical follow-up of the dogs is carried out six months post-injection. 87% of the cases showed satisfactory change following the treatment.

FIG. 11

An arthrosic dog as having a thrombocytopenia is treated with 1·10⁷ of NSC of phenotype MHC-I^{L}/CD90^{H} cryopreserved and thawed at ambient temperature. The cells are injected by intravenous perfusion. The results show a normalisation of the platelet concentrations over 3 months. The treatment also made it possible to overcome corticoid treatments.

FIG. 12: Examples representative of the different cytometric profiles and their associated characteristics.

NSC are isolated from canine placenta and amplified over several passages (2 to 5). Several NSC samples are analysed in flow cytometry in order to evaluate the CD90 (FL2-PE) and MHC-I (FL4-APC) expression. According to the phenotypic profile CD90/MHC-I, the NSC can be classed into several categories according to their biological characteristics and their composition. The setting up of the analysis matrix is shown in FIG. 18.

FIG. 13: Heterogeneity of MHC-I and CD90 expression among the amplified NSC

NSC are isolated from canine placenta, equine umbilical cord blood and equine umbilical cord matrix (respectively n=11, 13 and 18). The NSC were amplified at least over a passage and analysed in flow cytometry in order to evaluate their percentage of positivity and the rMFI (“relative mean of fluorescence intensity”) for MHC-I and CD90. This figure shows the disparities in markings and expression for these two markers among the NSC coming from the same source.

FIG. 14: Example representative of the fluctuations in positivity according to different thresholds for low marker expressions.

NSC are isolated from canine placenta, amplified and analysed by flow cytometry. CD90 expression is evaluated by single marking with FL2 (Fluorescence PE). The positivity thresholds are placed at about 2 (CD90pos_2SD) or 3 (CD90pos_3SD) standard deviations from the isotypic PE population, i.e. respectively at 4.5% or 0.3% of the isotypic PE population. This figure shows the amplitude of the results according to the two thresholds established for the same population of NSC with low CD90 expression (between 89.0 and 65.3%).

FIG. 15: Disparity in the proliferative potential among the populations of NSC coming from canine placentas.

NSC are isolated from canine placenta and amplified over several passages until a doubling time greater than 100 h is obtained. At each passage the number of cell doubling and the time for cell doubling is calculated according to Example C, part 1. The maximum number of doubling is then determined over the entire amplification period. The average of the doubling time is taken over 3 passages between P2 and P4 in such a way as to obtain data representative of the proliferation potential of the NSC.

FIG. 16: Flow chart of the method for manufacturing NSC MHC-I^{L}/CD90^{H} for therapeutic use—method of manufacturing assisted by analysis matrix.

Flow chart make it possible to determine the MHC-I/CD90 analysis matrix, collect the analysis data and allows for a double control that conditions the industrialisation of therapeutic units and the use thereof. The “True” condition implies that a population of NSC is qualified as MHC-I^{L}/CD90^{H} according to the invention.

FIG. 17: Flow chart of the method for manufacturing NSC MHC-I^{L}/CD90^{H} for therapeutic use—method of manufacturing with conditional analysis.

Flow chart allowing for a double control that conditions the industrialisation of therapeutic units and the use thereof. Contrary to the method of manufacturing assisted by analysis matrix (FIG. 16), the characterisation of the cells is based on a conditional analysis: if the population of NSC analysed is heterogeneous, the proportion of the sub-population MHC-I^{L}/CD90^{H} can be evaluated using an analysis matrix. If the population is homogeneous, the population must meet the threshold conditions.

FIG. 18: Examples representative of the establishment of analysis matrices of NSC with the MHC-I and CD90 doubling marking tool.

NSC are isolated from canine placenta or thawed in an early passage and amplified over several passages. The cells are analysed by MHC-I and CD90 double marking. Here the two examples show an individualisation of the sub-populations MHC-I^{L}/CD90^{H} and MHC-I^{H}/CD90^{L} during amplification. The two examples made it possible to establish an analysis matrix for the quantification of these two types of sub-populations of NSC. The NSC oriented towards a phenotype MHC-I^{L}/CD90^{H} are considered as able to be industrialised (NSC-2).

### EXAMPLES

### Example A: Method for Obtaining a Population of NSC

1. Isolation of the NSC from Canine Placenta

The canine extra-embryonic annexes (placenta, umbilical cord) are aseptically sampled during caesareans practiced in gestating dogs at term. As soon as the new-born puppy is removed from the amniotic sac and placed in safety, the extra-embryonic tissue is immediately transferred to a transport box containing a buffered saline solution with Dulbecco's phosphate (D-PBS) to be transported to the laboratory. The treatment of the extra-embryonic tissue is carried out at most within 48 h following sampling. All of the treatment steps of the tissue are carried out in a controlled environment, under a biosafety cabinet (BSC).

The tissue is transferred to a 100 cm2 Petri dish and the residual amniotic membrane is mechanically removed by dissection. The placenta is placed embryonic face against the plastic surface of the box and the uteroverdin present on the face of maternal origin is separated from the placenta by scraping the tissue. The placenta is rinsed 3 to 5 times in successive baths of D-PBS. The blood vessels and the umbilical cord are then mechanically removed from the placenta. The placental tissue is dissected into fragments of about 10-20 mm² then subjected to an enzymatic digestion by incubating the tissue fragments in a solution composed of DMEM (Dulbecco's modified Eagle medium) containing 0.5-4 mg/ml of type I collagenase, and more specifically a concentration of 1 mg/ml of type I collagenase. The enzymatic digestion takes place at 37° C. for 1 h but a digestion comprised between 30 min and 16 h can be carried out by decreasing the incubation temperature (ambient temperature (18-22° C. or 4° C.). At the end of the digestion, the enzymatic activity is stopped by dilution, by adding DMEM containing at least 10% foetal bovine serum (FBS) in a quantity equivalent to the solution of enzymatic digestion. The solution is then filtered over a 70-100 μm screen. The recovered cells are centrifuged at 800 g for 10 min. The cell button containing the neonatal stromal cells is rinsed with DMEM and again centrifuged at 800 g for 10 min. The cell button is taken in culture medium constituted of DMEM, 10% FBS, 2 mM glutamine and from 0 to 20 ng/ml of fibroblast growth factor (FGF). The cells are counted and seeded in culture dishes at a density comprised between 10⁴ and 2·10⁴ cells/cm². The cells are then cultivated in the culture medium described hereinabove in a controlled atmosphere at 37° C. and containing 5% CO₂. The medium is changed after 48 h then every 2-3 days. The cells are passed when the confluence reaches 70-80%.

2. Isolation of NSC of Equine Umbilical Cord Blood

The equine umbilical cord blood or placenta blood (PLB) is recovered during foaling. It is carried out in the most aseptic manner possible by puncture of the blood of the umbilical cord at umbilical vein using a needle connected to a blood sampling bag. The bags of PLB are transported to the laboratory under controlled temperature conditions (4-12° C.) and must be treated within 48 h following sampling. All of the treatment steps of the sample are carried out in a controlled environment, under a biosafety cabinet (BSC).

The PLB is transferred into a sterile container and diluted by half with D-PBS or any other physiological solution. The PLB thus diluted is deposited on a Ficoll solution (1.077 g/ml) contained in a tube, at a rate of 2 volumes of blood diluted to half for 1 volume of Ficoll. The tubes are centrifuged for 20-45 min at 700-1,000 g without a braking step. The PLB is then separated by density gradient and a cell ring is formed at the interphase between the diluted plasma and the Ficoll. The cell ring is recovered by aspiration and washed with D-PBS in a final volume of 50 ml. The cells are then centrifuged between 300-500 g for 5 to 10 min. Lysis of the erythrocytes can be carried out by incubating the cell button with the erythrocyte lysis buffer for a few minutes (3-10 min). Add to 50 ml with D-PBS. The cells are centrifuged between 300-500 g for 5 to 10 min. The cell button containing the neonatal stromal cells is taken in DMEM containing 2 mM of glutamine, 10% FBS and 0-20 ng/ml of FGF (amplification medium). The cells are counted and seeded in culture dishes at a density comprised between 10⁵ and 2.5·10⁵ cells/cm². The cells are then cultivated in an amplification medium in a controlled atmosphere at 37° C. and containing 5% CO₂. The medium is changed after 48 h then every 2-3 days. The cells are passed after the emergence of fibroblast colonies after 10-15 days.

3. Cell Passage and Amplification

At sub-confluence, the cells undergo a cell passage and optionally, an amplification procedure. The NSC are rinsed with D-PBS and treated with 0.05% of trypsin-EDTA for 2-5 min at 37° C. This makes it possible to detach the cells and to form a population of isolated cells. The cells are then taken with amplification medium constituted of DMEM, 10% FBS, 2 mM glutamine and from 0 to 20 ng/ml of fibroblast growth factor (FGF) and centrifuged between 300-500 g for 5 to 10 min. The NSC are taken in amplification medium, and counted via manual counting (trypan blue and Malassez cells) or using a cytometer. They are then seeded for 1,500 to 5,000 cells/cm² and cultivated on a plastic cell culture support in an amplification medium and in a controlled atmosphere at 37° C. and containing 5% CO₂. During the amplification process the cells can undergo between 0 and 15 cell passages.

4. Freezing and Cryopreservation of NSC

At the end of the first or second cell passage (P1-P2), the cells can be cryopreserved in seed units. To do this, after counting, the NSC are centrifuged between 300-500 g for 5 to 10 min and the cell button is taken in the freezing medium comprised either of DMEM medium enriched with 5-20% FBS and 5-10% (vol:vol) of DMSO or in a commercial cryopreservation medium, containing or not a fraction of DMSO. The cell concentration is comprised between 1·10⁶ and 15·10⁶ cells per ml of freezing medium. The freezing of the cells is carried out in controlled falling temperature conditions, using for example a CoolCell® Cell Freezing Container (BioCision) and by following the freezing procedure such as it is described by the manufacturer. The cells are then transferred for negative cold storage at temperatures comprised between −70° C. and −196° C.

The seed units can be used to generate cell units for therapeutic use. The seed units are thawed at 37° C. for 3-6 min and amplified in vitro. The cells are seeded in the culture medium at the density of 1,500-3,000 cells/cm2. The cells are amplified by successive passage in vitro. When a significant number of cells is produced (for example >150·10⁶ cells), the cells are frozen according to the protocol described hereinabove. The cells are distributed into hermetically sealed bottles with seals at a rate of 1·10⁶-15·10⁶ cells/ml in a freezing medium free of product of animal origin (such as for example the cryopreservation medium Recovery™ Cell culture freezing medium (Thermo Fisher), Cryostem™ freezing medium (Biological Industries). The bottles are lowered in temperature according to a controlled falling temperature protocol, at a rate of −1° C./min to −80° C. The bottles are then transferred at −80° C. for storage. Once obtained, the population of NSC is characterised on the one hand by its structural characteristics (presence/absence of markers) and on the other hand by its functional characteristics (proliferation, differentiation etc.).

### Example B: Structural Characterisation of the Population of NSC

1. Cytometric Analysis of NSC

Cytometric analysis aims to determine the presence of membrane markers on the surface of the NSC in particular CD11b, CD14, CD31, CD34, CD45, HLA-DR and more precisely to determine the expression rates of MHC-I and of CD90, thanks to the use of a specific antibody panel of each marker.

After isolation of the cells and during the amplification period, NSC at the passage P2 to P7 cultivated in a T75 are rinsed with 10 ml of D-PBS. 5 ml of trypsin/EDTA are then added and the cells are incubated 2 min at 37° C. The cells are detached and recovered in a 15 ml tube. The volume is adjusted to 15 ml by adding amplification medium. The cells are centrifuged 10 min at 300-500 g. The supernatant is aspirated then the cell button is taken in 2 ml of amplification medium. 40 μl of the suspension is diluted with 40 μl of trypan blue. The dilution is then supplemented with deposition on a counting slide and acquisition by a cell counter of the Luna type.

Generally between 10⁵-5·10⁵ cells are recovered for the cytometric analysis. In particular, 3 samples of 2·10⁵ cells are transferred into 1.5 ml Eppendorfs. The cells are taken in 1 ml of D-PBS and centrifuged at 500 g for 5 min. A second washing is carried out in the same experimental conditions. After elimination of the supernatant, the cells of the three tubes are respectively taken in 1 volume of 30-100 μl, in particular 50 μl, of marking buffer. This buffer is comprised of D-PBS and of 0.5-1% (v/v) of bovine serum albumin (BSA) or of 0.5-2% (v/v) of foetal bovine serum. 2.5 μl of a primary antibody coupled or not with a fluorochrome and that specifically targets the membrane marker of interest is added to the 50 μl of marking buffer.

The optimum concentration of antibodies used for the marking must be determined beforehand by those skilled in the art. The incubation required for the marking must also be determined by those skilled in the art and comprised between 15 min and 10 h at 4° C. in a dark place. In particular, the cells are incubated 20 min at 4° C. in a dark place.

A marking with a secondary antibody that targets the primary antibody can be carried out, after washing with D-PBS, in the case where the primary antibody is not directly coupled with a fluorochrome. Thus, in the case where the primary antibody is not coupled with a fluorochrome, 1 ml of D-PBS is added and the cells are centrifuged 5 min at 500 g. The supernatant is eliminated and 50 μl of marking buffer (D-PBS/2% of BSA) comprising 1 μl of secondary antibody solution targeting the primary antibody and marked by a phycoerythrin fluorochrome (PE) is added. The cells are incubated 20 min at 4° C. in a dark place. Then 1 ml of marking buffer is added and the cells are centrifuged 5 min at 500 g. The cells are taken in 200 μl of marking buffer.

Isotypic controls adapted to each marking have to be used as a negative control. Following the incubation with the antibodies, the cells are washed with D-PBS, centrifuged 5-10 min at 500 g and taken in a volume from 100 to 250 μl of marking buffer for analysis with the flow cytometer (Accuri C6, BD Biosciences).

2. Selection Based on MHC-I and/or CD90 Expression

a. Cytometric Analysis on Antibodies Validated with Defined Positivity/Negativity Thresholds

The cytometric analysis is carried out in such a way as to guarantee a signal coming from the fluorescence of the antibodies coupled or not with a fluorochrome which are potentially specifically fixed on the epitopes of interest (belonging to CD90 or to the MHC-I). To do this, those skilled in the art will have to correctly and carefully adjust the photomultipliers and the fluorescence compensations. Moreover the careful selection of the cell population to be analysed has to be carried out. It must on the other hand use non-marked control samples, use control samples marked with suitable total IgG coupled with the fluorochromes of interest and this in order to guarantee that the signal measured on the analysed samples takes account both of the auto fluorescence of the cells and the non-specific bonds of the antibodies. A potential treatment of the cells by receptor blockers Fc can also be considered.

In order to determine the positivity/negativity thresholds of the cells, the correct respective isotypic controls of each antibody coupled with their fluorochrome must be carefully selected.

In FIG. 1, four populations of NSC were analysed in cytometry. An analysis of the non-marked cells is conducted first of all. An analysis window based on the analysis parameters FSC-A/SSC-A is defined to select the events that correspond to the cells and eliminate the cell debris. Then an analysis window based on the analysis parameters FSC-H/FSC-A is defined for the analyse of the unique cells (elimination of cell dipoles) (FIG. 1A).

The tube marked with a primary isotype is analysed according to the preceding parameters. The PE marking is visualised on the suitable fluorescence channel corresponding to the fluorescence of interest of the cytometer (in FIG. 1B, the channel used is FL2). The analysis is carried out in the form of a 2D representation (bar chart with 2 parameters) based on the analysis of the fluorescence coming from the fluorochrome used (FL:2 for PE) and of the autofluorescence of the cells by using a fluorescence channel with an excitation/emission spectrum that is not used in the analysis (FL-4) (bar chart FL/event counting). The positivity threshold is placed in such a way that less than 0.5% and more than 0.1% of the cells are considered as positive. This same approach is carried out for each one of the markers MHC-I (FIG. 1C) and CD90 (FIGS. 1D and 1E). The 4 samples analysed have variable but homogeneous expressions of MHC-I (FIG. 1C) while the analysis of CD90 reveals two separate populations within the samples of NSC (FIG. 1D), for this latter marking it is thus possible to determine the proportion of cells with or without CD90 expression. The populations NSC-1 and NSC-2 meet the selection criteria for an MHC-I expression less than 10% and a CD90 expression of more than 80%. Because of this NSC-1 and NSC-2 are considered as MHC-I^{L}/CD90^{H} while the populations NSC-3 and NSC-4 are considered as MHC-I^{H}/CD90^{L}.

b. Variations in the Size/Auto Fluorescence and 2D Representation

It is important to keep all of these parameters fixed for the analysis of the populations of NSC of interest in order to determine the proportion of NSC with or without marker expression.

It is possible that variations in the size of the populations of NSC can be observed. Thus, the selections of analysis windows or “gates” of the populations to be analysed must not vary between the samples. However, in the case where a population heterogenous in size hinders the cytometric analysis, a separation in silico of the various sub-populations homogeneous in size can be considered. In this latter case, new positivity/negativity thresholds are to be determined for each sub-population by those skilled in the art.

It is possible that in parallel of a variation in size, a variation in the autofluorescence of the cells can be observed. In the latter case, a mode for representing the results over two acquisition channels (e.g.: FL-2/FL-4) is required in order to finely define the variations in expression of the markers between the different sub-populations emitting variable levels of autofluorescence. Here again, the determining of new positivity/negativity thresholds may need to be carried out by those skilled in the art. It is however to be noted that in the case where the population of NSC analysed has a morphology or autofluorescence that is excessively far from the original populations, the latter can be considered as non-compliant and therefore excluded.

c. Example of Determining Maximum Relative MFI Thresholds for MHC-I with the Use of Two Different Secondary Antibodies

“Maximum relative MFI (rMFI) threshold” means the relative limit MFI value that makes it possible to retain, for the industrialisation procedure, all the strains of NSC that have an rMFI less than this value.

Based on the configuration and on the analysis presented in section B. 2 a) hereinabove, several samples of NSC are analysed.

Seven populations of NSC coming from seven different placentas were analysed from a proliferative standpoint. Two among them allowed for an amplification greater than 20 cell doublings. These cells were analysed between P3 and P4 according to the procedure described in example B, part 1).

By way of examples we provide details hereinbelow on the determining of two maximum rMFI thresholds for two different immunomarking strategies (FIG. 2A and FIG. 2B). The characteristics of the antibodies used for these two strategies are listed the table 2 hereinbelow.

TABLE 2
Characteristics of the antibodies used

| Reference | Supplier | Type | Target | Coupled | Fluorochrome |
|---|---|---|---|---|---|
| Mouse (BALB/c) | AbSerotec | Primary | Isotype | yes | PE |
| IgG1, K MOPC-21 | | | | | |
| Mouse | Monoclonal | Primary | Isotype | no | — |
| COL2002/COLIS205C | Antibody | | | | |
| IgG2a | Center | | | | |
| | Washington | | | | |
| | State | | | | |
| | University | | | | |
| Mouse DG- | Monoclonal | Primary | MHC-I | no | — |
| BOV2001/DG-H58A | Antibody | | | | |
| IgG2a | Center | | | | |
| | Washington | | | | |
| | State | | | | |
| | University | | | | |
| CD90 Antibody | Bio Rad | Primary | CD90 | yes | PE |
| YKIX337.217 | | | | | |
| Rabbit F(ab′)2 anti | AbSerotec | Secondary | Mouse | yes | PE |
| mouse IgG STAR12A | | IgG | | | |
| Goat F(ab′)2 anti | EBioscience | Secondary | Mouse | yes | APC |
| mouse IgG secondary | | IgG | | | |

The isotype used is a mouse isotype e-COL2002/COLIS205C primary IgG2a non-coupled to a fluorochrome (Monoclonal Antibody Center Washington State University). An anti-MHC-I canine antibody of mouse type DG-BOV2001/DG-H58A IgG2a was used for the primary marking of the MHC-I (Monoclonal Antibody Center Washington State University).

Two types of secondary rabbit antibodies F(ab′)2 anti mouse IgG STAR12A (AbSerotec) and goat t F(ab′)2 anti mouse secondary IgG (eBioscience) coupled respectively with a fluorochrome of the PE (FIG. 2A) and APC (FIG. 2B) type were used for this study and made it possible to determine two maximum thresholds of rMFI specific to each secondary antibody.

In accordance with part Example B. 2)a) of the present application, the cells marked by the isotype or by the MHC-I are analysed in the Accuri C6 cytometer. The channels used for the analysis of the fluorescences emitted correspond to the channel FL-2 for the rabbit antibody F(ab′)2 anti mouse IgG STAR12A (AbSerotec) and FL-4 for the goat antibody F(ab′)2 anti mouse secondary IgG (eBioscience). For each antibody and each strain of NSC (market with the isotype and MHC-I), the values of the MFI are extracted. The rMFI of each sample marked with MHC-I are calculated by normalisation of the values of MFI of the marked cells with respect to those of the cells incubated with the isotype (marked MFI/isotype MFI).

The analysis of the MHC-I expression indicates variable expressions of this marker between the different samples. The analytical method proposed consists of normaliser the average fluorescence values (MFI; Mean Fluorescence Intensity) of the cells marked with the antibody of interest (here MFI FL2_{(MHC-I-PE)}) with the MFI of the cells marked with the isotype coupled with the same fluorochrome (here MFI FL2_{(isotype-PE)}). A ratio (rMFI) is thus calculated MFI FL2_{(MHC-I-PE)}/MFI FL2_{(isotype-PE)}.

For each secondary antibody used:
the highest rMFI value of the strains meeting the criteria of minimum number of doubling, positive chondrogenesis, negative osteogenesis and immunomodulation positive is taken for the calculation of the maximum threshold for rMFI;
and the lowest rMFI value of the strains of which the number of doubling is less than 20 is also retained.

Thus, for the rabbit antibody F(ab′)2 anti mouse IgG STAR12A (AbSerotec) marked PE the average of these two values of rMFI is calculated and makes it possible to determine a maximum threshold of rMFI of 2.5 (FIG. 2A). For the goat antibody F(ab′)2 anti mouse secondary IgG (eBioscience) marked APC, the average of these two values of rMFI is calculated and makes it possible to determine a maximum threshold of rMFI of 10 (FIG. 2B).

d. Example of Determining CD90 Acceptance Thresholds

The same approach described in section 2) c). can be carried out to determine the minimum threshold for rMFI for CD90 that makes it possible to exclude the cells that have an CD90 expression rate that is too low. The analysis of the CD90 in the NSC, the latter is expressed substantially heterogenous in the different populations of NSC. Contrary to MHC-I, the loss of expression of CD90 is materialised by the emergence of a separate sub-population. Two expression levels of CD90 can thus be observed, one low expression (CD90^{L}) and one high expression (CD90^{H}). Therefore, the two normal populations revealed do not always make it possible to determine a single value for MFI.

e. Example of Determining Acceptance Thresholds for MHC-I and for CD90 Via a Double-Marking Strategy on a Population of Heterogenous NSC.

In order to limit the constraints linked to the heterogenous populations for the determining of thresholds, the same approach described in section 2) c). can be carried out to determine the minimum rMFI threshold for CD90 and maximum for MHC-T and this from isolated heterogenous (mixed) populations. Among 14 populations of NSC analysed with double marking CD90 and MHC-I during cell amplification, only one population of NSC did not pass through a heterogenous population stage regarding CD90 and MHC-I. Over the other 13, a stage of mixed population (i.e. represented by the coexistence of two populations MHC-I^{H}/CD90^{L} and MHC-I^{L}/CD90^{H}) was able to be observed between P1 and P4. These two sub-populations have always shown differences in expressions of the two markers, revealing a population of interest MHC-I^{L}/CD90^{H} and another population MHC-I^{H}/CD90^{L}. The analysis of the rMFI of the two respective sub-populations MHC-I^{H}/CD90^{L} and MHC-I^{L}/CD90^{H} makes it possible to establish discriminations thresholds in order to identify the population of interest MHC-I^{L}/CD90^{H} (FIG. 3). Regarding the MHC-I, the threshold that makes it possible to identify a population MHC-I^{L}/CD90^{H} and that minimises false positives is a maximum rMFI of 15. For CD90, the minimum rMFI is 20.

### Example C: Biological Characterisation of the NSC Population

1. Evaluation of the Proliferative Activity of NSCs

The proliferation of NSCs is evaluated for 7 to 8 consecutive cell passages (if attainable). With each cell passage (once a week; or every 6 to 8 days), the cells are detached from their culture substrate by means of trypsin/EDTA 0.5% for 2-3 minutes. Culture medium is added and the cells are centrifuged 5 min/300 g. The cell pellet is returned to a defined volume of culture medium and the cells are counted by a trypan blue exclusion test by means of an electronic counter. The doubling number is calculated with each cell passage according to the following formula: Nb of doublings=LOG (Nf/Ni)/LOG(2) (Nf: number of final cells and Ni: number of initial cells). The total number of cell doublings is equal to the sum of the number of cumulated doublings with each cell passage. The cell amplification is stopped as soon as the number of doublings is below 1, which means that the cell population is no longer capable of doubling. The proliferative activity of a population of MHC-I^{L}/CD90^{H} cells is compared with that of a population of MHC-I^{H}/CD90^{L} cells (FIG. 4).

A cell population of NSCs is considered to be acceptable for industrialisation if the total number of consecutive cell doublings is greater than or equal to 20.

As shown in table 3, the cells of phenotype MHC-I^{L}/CD90^{H} have a total number of consecutive cell doublings greater than 20, contrary to MHC-T^{H}/CD90^{L} cells. This proves that the NSCs of phenotype MHC-I^{L}/CD90^{H} have a proliferative activity which is much greater than the MHC-I^{H}/CD90^{L} cells, which enables them to be used for industrialisation contrary to the MHC-I^{H}/CD90^{L} cells.

2. Chondrogenic Differentiation

The chondrogenic differentiation capacity of the NSCs is verified by incubating the cells in a specific differentiation medium of this differentiation method.

To achieve this, the NSCs are detached from their plastic substrate by trypsination and counted. The cells are then used to form micromasses by gravitation in a droplet of amplification medium. To achieve this, a solution of approximately 7·10⁶ cells/ml of amplification medium is then prepared. 35 μl of this correctly homogenised solution is then deposited in droplet form onto an untreated and uncoated plastic surface. This surface is then returned to incubation for 24 h in a humid atmosphere at 37° C. and 5% CO₂. By way of example, the cover of a Petri dish returned to its stand containing D-PBS can be used as a culture substrate.

After 24 h incubation, a micromass of NSCs forms at the base of the droplet. The micromass is recovered and transferred to a 6 well plate so as to deposit 5 micromasses per well. 2 ml chondrocyte differentiation medium are then added per well in order to induce the chondrogenic differentiation of the NSCs. This medium is composed of DMEM 4.5 g/l glucose, insulin-transferrin-selenium 1X, dexamethasone 0.1 μM, sodium pyruvate 1 mM, 50 μg/ml ascorbic acid-2-phosphate, 40 μg/ml L-proline, 10 ng/ml TGF-β3 (or a combination of 10 ng/ml TGF-β1 and 50 ng/ml BMP-2). The chondrogenic differentiation is performed within 7 days by changing the differentiation medium every 2-3 days. Acquisition by photonic microscopy is performed at the end of the culture by a ×4 magnification Nikon TS2 coupled to a TS2-P-CF camera.

Three populations of NSCs were analysed: NSC-1, NSC-2 and NSC-3. Population NSC-1 is an MHC-I^{L}/CD90^{H} phenotype, whereas the NSC-2 and NSC-3 populations are an MHC-I^{H}/CD90^{L} phenotype.

At the end of the culture, the micromasses observed using the microscope exhibit a substantial difference in size (FIG. 5). The surface occupied in the image by the micromasses is analysed by ImageJ and expressed in pixels². The MHC-I^{L}/CD90^{H} NSCs form a chondrogenic micromass with a larger area than the MHC-I^{H}/CD90^{L} NSCs (FIG. 5A and FIG. 5B).

The medium is then removed from the wells and the neo-tissue and used for RNA or protein extraction in order to analyse the expression of specific markers of the chondrogenic lineage such as type II collagen, aggrecan, COMP, SOX9.

The total RNA are extracted by crushing the micromasses by means of a plastic pestle in the presence of TRIzol Reagent (Sigma). The remainder of the extraction procedure is performed according to the specifications of the manufacturer. The RNA are retro-transcribed using a PrimeScript Reverse Transcriptase (Clonetech) according to the specification of the manufacturer. The RTqPCR analysis is performed by means of an Mx3000p thermocycler as well as MxPRO software (Stratagene). The results are expressed in the form of a relative rate of expression of the target genes relative to a housekeeping gene (of the type Gapdh) by means of the 2-ΔΔCT method.

The expression of the Col2a1 gene was analysed by RTqPCR and normalised by the expression of the Gapdh gene. This expression of the Col2a1 gene was tracked in the micromasses obtained for each cell population (NSC-1, NSC-2 and NSC-3) after 7 days of chondrogenic differentiation (treated) relative to this same cell population which has not been subjected to chondrogenic differentiation (Ctrl). The data are expressed in a relative expression of Col2a1 relative to the control (FIG. 5C).

We consider a relative expression value of Col2a1 relative to undifferentiated cells cultivated on a plastic substrate greater than 100 to be acceptable.

The expression of these markers makes it possible to conclude a chondrogenic differentiation potential of the NSCs.

FIG. 5D shows that the expression of the Col2a1 gene in the NSC-1 population of phenotype MHC-I^{L}/CD90^{H} is higher than in the NSC-2 and NSC-3 populations which are of phenotype MHC-I^{H}/CD90^{L}. This makes it possible to conclude a greater chondrogenic differentiation potential in the MHC-I^{L}/CD90^{H} NSCs.

In analysis of 8 populations of NSCs, the results have shown a difference in the expression of Col2a1 between the MHC-I^{H}/CD90^{L} and MHC-I^{L}/CD90^{H} NSCs (FIG. 5D).

3. Osteogenic Differentiation

The low osteogenic differentiation capacity of NSCs is verified by incubating the cells in a specific differentiation medium for this differentiation method.

To achieve this, the NSCs are detached from their plastic substrate by trypsination and counted. The NSCs are seeded in a density between 1500 and 5000 cells/cm² in a 6 well plate in an amplification medium under a controlled atmosphere at 37° C. and containing 5% CO₂. When the cells achieve 50-75% confluence, the proliferation medium is removed and replaced by osteogenic differentiation medium composed of DMEM, 10% SVF, 2 mM glutamine, 0.1 μM dexamethasone (Sigma), 50 μM ascorbic acid 2-phosphate (Sigma) and 10 mM β-glycerophosphate (Sigma). The medium is renewed 2 times/week, for a period between 10 and 15 days.

Following the differentiation process, the wells are washed with D-PBS and the cells are fixed for example by a 10% neutral buffered formalin solution for 1 h minimum. Dyeing is carried out with a 1% Alizarine red solution (weight/volume) to show the presence of calcic deposits. The wells are then rinsed with H₂O and the dye is analysed under the microscope.

The absence of calcic deposits makes it possible to determine the absence of osteogenic differentiation potential.

4. Evaluation of the Immunomodulatory Potential of NSCs by PGE2 Expression

The potential of NSCs to express molecules exerting an immunomodulatory effect is evaluated by dosing the molecules secreted by the NSCs of phenotype MHC-I^{L}/CD90^{H} in basal condition or after stimulation with a proinflammatory cytokine.

To achieve this, the MHC-I^{L}/CD90^{H} NSCs seeded at 5·10⁴ cells/cm² are cultivated in proliferation medium as described above for 72 h or in a medium complemented with 5 ng/ml gamma interferon. At the end of this incubation, the culture medium is centrifuged 10 min/500 g. The supernatant is frozen at −80° C. The factors of interest are analysed by an ELISA test by following the individual instructions of each kit. The dosing of prostaglandin E2 (PGE2) is performed by means of the KGE004B kit (R&D System). The results in FIG. 6 reveal an increase in the expression of PGE2 when the NSCs are treated with a proinflammatory cytokine such as IFN, which demonstrates a potential of NSCs to express molecules, in particular PGE2, which can exert an immunomodulating effect.

5. Evaluation of the Immunomodulatory Potential of NSCs from their Antiproliferative Effect on the Lymphocytes

The capacity of the NSCs to inhibit the proliferation of lymphocytes in vitro is evaluated by co-cultivating the NSCs of phenotype MHC-I^{L}/CD90^{H} with blood mononuclear cells (PBMCs) in the presence of a mitogenic agent.

To achieve this, the PBMCs are isolated from a blood sample taken from a donor dog or a donor horse over a Ficoll gradient. The PBMCs are then incubated with a fluorescent dye (CellTrace CFSE, Thermo Fisher) which makes it possible to measure cellular proliferation over several generations. 0.2·10⁶ PBMCs marked by Celltrace are added to the wells of a 96 well plate in which the NSCs were seeded the day before in a concentration (2·10⁴ NSC/wells); thus making it possible to obtain a ratio of NSC:PBMC equivalent to 1:10. The NSCs are treated with mitomycin (10 μg/ml for 1.5-2 h at 37° C.) and rinsed 3 times in culture medium before the addition of PBMCs. The proliferation medium of the lymphocytes is added (RPMI, 10% SVF, 2 mM glutamine, 10 mM hepes, 50 μM β-mercaptoethanol, 5 μg/ml concanavalin A). The PBMC/NSC co-cultures and the control cultures (PBMC without NSCs) are incubated for 4 days in an incubator at 37° C. Following the culture, the non-adherent cells are recovered from the wells, centrifuged and washed in D-PBS. The cells are then marked with an anti-CD3 antibody coupled with a FITC fluorochrome for 30 min at 4° C. The cells are then washed in D-PBS before flow cytometry analysis (Accuri C6, BD Biosciences). The cytometric analysis consists of evaluating the Celltrace signal within the viable CD3+ population. A proliferation index (PI) is calculated by using analysis software, such as for example Modfit®. The proliferation index of PBMCs cultivated in the presence of a mitogenic agent without NSC (PI ctrl) is fixed arbitrarily at 1. The proliferation index of the PBMCs cultivated in the presence of mitogenic agent and NSCs in a ratio 1:10 (PI 1:10) is standardised relative to (PI ctrl). The lineage of NSCs is considered to exert a significant antiproliferative effect if the ratio (PI 1:10)/(PI ctrl) is less than or equal to 0.5. The significant reduction of the proliferation index in the cocultures with the NSCs in FIG. 7, shows that the NSCs of phenotype MHC-I^{L}/CD90^{H} exert antiproliferative activity on the lymphocytes. The NSCs therefore have an immunomodulatory potential in vitro, and in this case an immunosuppressant potential.

### Example D: Evaluation of the Biological Properties of Cryopreserved NSCs

The biological activity (viability, proliferative activity, immunomodulatory potential) of NSCs of phenotype MHC-I^{L}/CD90^{H} is evaluated in vitro and compared with the properties of the NSCs kept in culture.

The NSCs cryopreserved at −80° C. for several months are thawed to ambient temperature for 8-10 minutes. The cell suspension in its cryoprotectant medium is transferred into a 15 ml Falcon tube. An aliquot (50 μl) is taken to perform trypan blue staining. The sample prepared in this way is analysed by means of an electronic counter which estimates the cell viability from the ratio of the number of cells stained by trypan blue/total number of detected cells. In an alternative manner, an aliquot of 50 μl cell suspension is taken and mixed with 50 μl of a propidium iodide solution (PI; 10 μg/ml). The solution prepared in this way is immediately analysed by a flow cytometer. The cells having a signal on the detection channel of the PI correspond to dead cells.

The capacity of the MHC-I^{L}/CD90^{H} NSCs to be proliferated in vitro post-thawing is evaluated by seeding a defined quantity of cells onto a culture substrate in proliferation medium for 7 days. The proliferative activity of the NSCs is evaluated according to the method described above (paragraph 1).

The immunomodulatory potential of the cryopreserved NSCs is evaluated according to the method described in paragraph 5 of the examples.

The results of FIG. 8 show that the cell viability of the NSCs of phenotype MHC-I^{L}/CD90^{H} is greater than 80% for up to 12 months of preservation at −80° C. (FIG. 8A). The proliferative activity of the NSCs cryopreserved at −80° C. is not significantly modified compared to the NSCs kept in culture, as indicated by the number of doublings and the time of cellular doubling which are similar regardless of the storage period at −80° C. (FIG. 8B). The immunomodulatory activity of the cryopreserved NSCs is maintained as shown by the results of the in vitro lymphocyte inhibition proliferation inhibition test (FIG. 8C and FIG. 8D).

### Example E: In Vivo Evaluation of the Therapeutic Effect of an Injection of Ready-to-Use Cryopreserved NSCs for Treating Osteoarthritis in a Dog

Clinical case 1 (Dog 1): English bulldog, 10 years old, presenting with dysplasia of two hips as well as a rupture of the anterior cruciate ligament along with chronic pronounced lameness and virtually permanent pain in the hind legs, characteristic of osteoarthritis. A treatment of intra-articular injection of cryopreserved NSCs taken from a canine placenta (from a different individual than the treated individual), is prescribed for each joint. Three units of 1·10⁷ cryopreserved NSCs are thawed 24 h prior to administration. The treatments are sent to the veterinary clinic by a temperature-controlled transporter (4-12° C.). Each of three units of NSCs are injected directly respectively into each of the hips and into the knee joint, without previously washing the cells. The progress of the animal is monitored by a questionnaire which has been developed and validated for evaluating the mobility of dogs (LOAD; Liverpool Osteoarthritis In Dog). This document is given to the animal's owners for evaluating in a semi-quantitative manner the progression of the lameness and comfort of the animal with 13 questions scored from 0 to 4. The score thus varies between 0 and 52, 0 corresponding to the score of a healthy dog and 52 corresponding to the highest score for pain and discomfort.

Before treatment, the score is evaluated to be 41/52, which corresponds to joint pain/discomfort which can be considered extreme. Three months after injection, the score given by the owners is evaluated as 28/52, which indicates a positive progress in the movement of the animal of 32%.

Clinical case 2 (Dog 2): Yorkshire, 10 years old, presenting with pronounced lameness in the elbow and diagnosed with elbow dysplasia with fragmentation of the medial coronoid process associated with arthritic lesions. Treatment by intra-articular injection of cryopreserved NSCs taken from a canine placenta (from a different individual than the treated individual), is prescribed for the elbow. A unit of 1·10⁷ cryopreserved NSCs is thawed 24 h prior to administration. The treatment is sent to the veterinary clinic by temperature-controlled transporter (4-12° C.). The unit of NSCs is injected directly into the elbow, without previously washing the cells. The efficacy of the treatment is evaluated 2 months post-treatment by means of a clinical evaluation carried out by the veterinary and by the LOAD questionnaire filled out by the owners. The clinical evaluation of the animal consists of attributing a score based on an examination of the mobility of the animal. The following criteria are evaluated: lameness (1-5), pain on palpation (1-3), swelling of the joint (1-3), cracking (1-3); or a total score of 14 (14 corresponding to the most critical state).

Before treatment with NSCs, the clinical score attributed to the animal is 10/14 (lameness 3/5 (moderate); pain on palpation 3/3 (severe), swelling of the joint 2/3 (moderate) and cracking at 2/3 (moderate). The owner's score before treatment is evaluated as 34/52.

The results 2 months post-injection show a clinical score of 6/14; either a clinical improvement of 59% and an owner's score of 20/52; or an improvement in mobility of 41%.

FIG. 9 shows the progression of the LOAD score of each animal before treatment and post-administration of cryopreserved NSCs (dog H: 3 months post-injection/dog D: 2 months post-injection).

### Example F: Evaluation of the Progress of the Mobility of Dogs Suffering Osteoarthritis Following the Administration of a Preparation of Ready-to-Use Cryopreserved NSCs

Fifteen dogs suffering from articular osteoarthritis (knee, elbow, hip or knee joint) were recruited for an intra-articular injection of 1·10⁷ cryopreserved/joint NSCs. These NSCs were taken from a canine placenta (from a different individual than the treated individual). The product is thawed 24 h prior to administration, sent to the clinic by temperature-controlled transporter (4-12° C.) and injected without washing the cells.

Six months post-injection, a questionnaire is sent to veterinaries for evaluating the efficacy of the treatment. A score of 1 to 3 is attributed corresponding to very satisfactory progress (1); satisfactory progress (2); unsatisfactory progress (3).

The progress of the mobility of the animal is evaluated as satisfactory in 87% of cases (80% with progress considered to be very satisfactory); 13% of responses indicate unsatisfactory progress of the animal following the treatment (FIG. 10).

### Example G: In Vivo Evaluation of the Therapeutic Effect of an Injection of Cryopreserved NSCs on Canine Thrombocytopenia

Clinical case: Teckel (5 kg), 8 years old, presents at a consultation with ecchymoses on the abdomen. The medical history of this animal includes 2 episodes of thrombocytopenia at the age of 6 months and 6 years. During these two episodes, it was necessary to resort to corticoid treatment at an immunosuppressant dose, suggestive of an auto-immune cause of the disease. Blood tests are carried out, ruling out an infectious disease. Diagnostic imaging does not identify a neoplastic process. The blood count reveals a platelet level of 76·10³/mm³. The mean platelet volume (MPV) is 5.7 fl, close to the lower limit (5 to 12 fl). The number of neutrophiles is measured at 540/mm³, or 8.8% of the total number of leucocytes (6 000/mm³). The other constants are normal. In view of the medical history of the animal and the absence of an identified pathology, the diagnosis is therefore idiopathic or primary immune-mediated thrombocytopenia.

A corticoid and immunosuppressant-based treatment is prescribed which supports a temporary normalisation of the platelet count, with a recurrence at 3 months. A perfusion of 1·10⁷ cryopreserved NSCs is delivered intravenously. The NSCs come from a canine placenta (from a different individual than the treated individual). The cells are suspended in a perfusion of 50 ml NaCl and are administered slowly for a period of 25 minutes. Monitoring (heart and respiratory rate, rectal temperature, mucosa check) is carried out during the perfusion. Clinical monitoring is performed over a period of 3 months with platelet doses.

During the perfusion of NSCs, the animal does not show any clinical signs or side effects in the 72 hours following the perfusion. In the month following the administration of NSCs, the corticoid and immunosuppressant treatments are progressively reduced. One month after treatment with NSCs, the platelet concentration is evaluated as 200·10³/mm³. The corticoid treatment is suspended. Two months after treatment, the platelet concentration is evaluated as 255·10³/mm³, 3 months after treatment, the platelet concentration is evaluated as 460·10³/mm³.

The data show a normalisation of platelet concentrations up to 3 months post-injection without taking corticoids (FIG. 11).

### Example H: In Vivo Evaluation of the Therapeutic Effect of an Injection of Cryopreserved NSCs for Treating Chronic Inflammatory Bowel Disease

Clinical case: A female Shar Pei dog, 4 years old, diagnosed with chronic inflammatory bowel disease in February 2017. The dog has diffuse and moderate duodenal enteritis accompanied by lymphoplasmocytic infiltration. These different lesions cause vomiting, diarrhoea and chronic abdominal pain. The veterinary prescribes increasing doses of cortisone to determine the dose required to keep the animal stable. The dose of 2 mg/kg/day is selected. For 3 years after starting the treatment with cortisone, the owner consults numerous veterinaries with the aim of reducing the currently prescribed dose which may be harmful over the long term. None of the treatments made it possible to reduce the dose and the animal had to remain on the cortisone treatment permanently.

A treatment is prescribed of intravenous injection of cryopreserved NSCs. The NSCs come from a canine placenta (from a different individual than the individual being treated). A unit of 1·10⁷ cryopreserved NSCs is thawed 24 h before administration. The treatment is conveyed to the veterinary clinic by a temperature-controlled transporter (4-12° C.). The unit of NSCs is injected directly via a perfusion of normal saline, without previously washing the cells.

Three days after the administration of the cells, the veterinary reduces the dose of cortisone by half then at the end of 10 days again reduces the dose by half to obtain a dose of 1 mg/kg every 2 days, or a reduction of the dose of cortisone by 4. Twenty days after the treatment the dog remains stable and fully tolerates the reduction of cortisone.

### REFERENCES

Bruder S P, Jaiswal N, Haynesworth SE. Growth kinetics, self-renewal, and the osteogenic potential of purified human mesenchymal stem cells during extensive subcultivation and following cryopreservation. J Cell Biochem 1997 February; 64(2):278-94;
Dominici, M. L. B. K., Le Blanc, K., Mueller, I., and al., Minimal criteria for defining multPIotent mesenchymal stromal cells. The International Society for Cellular Therapy position statement. Cytotherapy, 2006, vol. 8, no 4, p. 315-317;
Phinney, D. G. (2012). Functional heterogeneity of mesenchymal stem cells: implications for cell therapy. Journal of cellular biochemistry, 113(9), 2806-2812);
Jacobs, Sandra A., Roobrouck, Valerie D., Verfaillie, Catherine M., and al., Immunological characteristics of human mesenchymal stem cells and multPIotent adult progenitor cells. Immunology and cell biology, 2013, vol. 91, no 1, p. 32-39;
Tessier, L., Bienzle, D., Williams, L. B., & Koch, T. G. (2015). Phenotypic and immunomodulatory properties of equine cord blood-derived mesenchymal stromal cells. PLoS One, 10(4);
Lepage, S. I., Lee, O. J., & Koch, T. G. (2019) Equine Cord Blood Mesenchymal Stromal Cells Have Greater Differentiation and Similar Immunosuppressive Potential to Cord Tissue Mesenchymal Stromal Cells. Stem cells and development, 28(3), 227-237;
Sarugaser, R., Lickorish, D., Baksh, D., Hosseini, M. M., & Davies, J. E. (2005). Human umbilical cord perivascular (HUCPV) cells: a source of mesenchymal progenitors. Stem cells, 23(2), 220-229;
Portmann-lanz, C. Bettina, Schoeberlein, Andreina, Huber, Alexander, and al., Placental mesenchymal stem cells as potential autologous graft for pre-and perinatal neuroregeneration. American journal of obstetrics and gynecology, 2006, vol. 194, no 3, p. 664-673;
Sibov, T. T., Severino, P., Marti, L. C., Pavon, L. F., Oliveira, D. M., Tobo, P. R., Campos, A. H., Paes, A. T., Amaro, E., F Gamarra, L., and al., (2012). Mesenchymal stem cells from umbilical cord blood: parameters for isolation, characterization and adPTogenic differentiation. Cytotechnology 64, 511-521.

1. Population of neonatal stromal cells (NSC), said population comprising neonatal stromal cells of low MHC-I phenotype (MHC-I^{L}).

2. Population of neonatal stromal cells according to claim 1, said population comprising neonatal stromal cells of MHC-I^{L} phenotype and high CD90 phenotype (CD90^{H}).

3. Population of neonatal stromal cells according to claim 1 wherein at least 80% by number of said population are MHC-I^{L} and wherein at least 80% by number of the cells of said population are CD90^{H}.

4. Population of neonatal stromal cells according to claim 1 wherein at least 80% of the cells of said population of NSC have a consecutive cell doubling capacity greater than 20 cumulative doublings.

5. Population of neonatal stromal cells according to claim 1 wherein at least 80% of the cells of said population of NSCs have:
a chondrogenic differentiation potential, and/or
an immunomodulatory potential.

6. Population of neonatal stromal cells according to claim 1 wherein the NSCs are taken from a neonatal tissue sample.

7. Population of neonatal stromal cells according to claim 1 wherein the sample is taken from the neonatal tissue or fluids of a mammal, in particular from a dog, cat, horse or human being.

8. Population of neonatal stromal cells for autologous, allogenic or xenogenic therapeutic use in the dog, cat, horse, or human being, for the treatment of:
a. tissue or joint damage, with or without an inflammatory component,
b. degenerative diseases, in particular osteoarthritis, tendinopathies, tissue fibrosis, Alzheimer's, Parkinson's disease,
c. auto-immune, inflammatory and/or infectious diseases, in particular atopic dermatitis, gingivostomatitis, thrombocytopenia, epidermolysis bullosa, sepsis, chronic inflammatory bowel diseases (CIBD);
d. transplant rejection, or
e. tumour diseases.

9. Pharmaceutical composition comprising a population of neonatal stromal cells according to claim 1 and a pharmaceutically acceptable vehicle.

10. Pharmaceutical composition according to claim 9, wherein the population of neonatal stromal cells comprises from 1·10⁶ to 1·10⁸ cells in 0.1 ml to 15 ml pharmaceutical composition.

11. Pharmaceutical composition according to claim 8 wherein said pharmaceutically acceptable vehicle is a solution comprising a cryoprotectant and characterised in that said composition is in frozen form.

12. Ready-to-use injectable solution comprising a unit dose of 1×10⁶ to 1×10⁸ of a NSC population as defined in claim 1 in solution with a cryoprotectant.

13. In vitro method for obtaining a pharmaceutical composition of neonatal stromal cells taken from neonatal tissue, said pharmaceutical composition comprising as an active ingredient a population of neonatal stromal cells comprising NSCs of phenotype MHC-I^{L}, and optionally of phenotype CD90^{H}, said method comprising:
a. providing one or more neonatal biological samples comprising NSCs, the biological sample or samples preferably having been obtained from one or more individuals,
b. isolating the population of NSCs present in the biological sample or samples,
c. optionally, at least one step of ex vivo amplification of the NSCs obtained in step b.,
d. optionally, cryopreserving the population of NSCs obtained in step b. or c.,
e. optionally, stimulating by physical, biological and/or chemical effector the population of NSCs obtained in step b., c. or d.,
f. characterisation of the presence of NSCs of phenotype MHC-I^{L}, and optionally of phenotype CD90^{H}, among at least 80% of the population of NSCs, after isolation in step b. and/or after the ex vivo amplification step of step c. and/or after the cryopreservation of the NSCs in step d.,
g. suspending the population of NSCs comprising at least 80% NSCs of phenotype MHC-I^{L}, and optionally of phenotype CD90^{H}, in a pharmaceutically acceptable suspension medium.

14. In vitro method for obtaining a pharmaceutical composition of neonatal stromal cells according to claim 12, wherein the neonatal biological sample or samples suppled in step a. are taken from a neonatal tissue sample, in particular from one or more placentas and/or one or more umbilical cords, or from a sample of neonatal fluid, in particular the blood of one or more umbilical cords and/or characterised in that the neonatal biological sample or samples supplied in step a. are taken from a mammal, in particular a dog, cat, horse or human being.

15. In vitro method for obtaining a pharmaceutical composition of neonatal stromal cells according to claim 12 wherein said method comprises an amplification step c., said amplification step comprising 1 to 5 cell passages and/or said amplification step corresponds to a doubling of the population of NSCs isolated in step b. of 2 to 25 cell doublings.

16. Population of neonatal stromal cells according to claim 6 wherein said neonatal tissue sample is selected from the group consisting of one or more placentas, one or more umbilical cords, and one or more neonatal fluids.

17. Population of neonatal stromal cells according to claim 16 wherein the one or more neonatal fluids are blood of one or more umbilical cords.

18. Pharmaceutical composition according to claim 9, wherein the population of neonatal stromal cells has a concentration between 5×10⁴ and 1×10⁹ cells/ml.
